(19) United States
(12) Patent Application Publication
(10) Pub. No.: US 2006/0034833 A1
(43) Pub. Date: Feb. 16, 2006

(54) Single domain antibodies directed against interferron-gamma and uses therefor

(76) Inventor: Els Beirnaert, Gent (BE)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/534,345

(22) PCT Filed: Nov. 7, 2003
(86) PCT No.: PCT/BE03/00194

§ 371 Date: May 9, 2005

(30) Foreign Application Priority Data
Nov. 8, 2002 (US) 60425063
Jan. 10, 2003 (EP) 03447005.4

(51) Int. Cl. C07K 16/24; A61K 39/395

(52) U.S. Cl. 424/133.1; 424/145.1; 530/388.23

(57) ABSTRACT

The present invention relates to polypeptides derived from single domain heavy chain antibodies directed to interferon gamma. It further relates to single domain antibodies that are Camelidae VHHs. It further relates to methods of administering said polypeptides. It further relates to protocols for screening for agents that modulate the IFN-gamma receptor, and the agents resulting from said screening.

### FIELD OF THE INVENTION

The present invention provides polypeptides comprising one or more single domain antibodies directed towards Interferon gamma (IFN-gamma). The present invention further relates to their use in diagnosis and therapy. Such antibodies may have a framework sequence with high homology to the human framework sequences. Compositions comprising antibodies to Interferon gamma (IFN-gamma) alone or in combination with other drugs are described.

### BACKGROUND

Interferon gamma (IFN-gamma) is believed to play an important role in various disorders, for example in inflammatory disorders such as rheumatoid arthritis, Crohn's disease, inflammatory bowel disease, ulcerative colitis, multiple sclerosis and hyperimmune reactions in the eye. IFN-gamma has also been shown to play a significant role in the pathology of autoimmune diseases. For example, the presence of IFN-gamma has been implicated in rheumatoid arthritis (Brennan et al, Brit. J. Rheum., 31, 293-8 (1992). Several strategies to antagonize the action of these cytokines have been developed and are currently used to treat various disease states.

Interferon gamma (IFN-gamma) in its bioactive form is a dimer and the interaction with the Interferon gamma (IFN-gamma) receptor occurs through interaction of two loops present on the homodimeric IFN-gamma with loop structures on the IFN-gamma receptor (Walter et al, nature, 376, 230-235 (1995)).

An Interferon gamma (IFN-gamma) inhibitor which has sufficient specificity and selectivity to IFN-gamma may be an efficient prophylactic or therapeutic pharmaceutical compound for preventing or treating inflammatory disorders. Methods of treating an autoimmune disease by means of an antibody to IFN-gamma have been described. Diseases include multiple sclerosis, rheumatoid arthritis, ankylosing spondylitis, juvenile rheumatoid arthritis, and psoriatic arthritis (U.S. Pat. No. 6,333,032 Advanced Biotherapy Concepts, Inc.). Other diseases include Crohn's disease and psoriasis (U.S. Pat. No. 6,329,511 Protein Design Labs). Yet other diseases are bowel disease, ulcerative colitis and Crohn's disease (EP0695189 Genentech).

Yet none of the presently available drugs are completely effective for the treatment of autoimmune disease, and most are limited by severe toxicity. In addition, it is extremely difficult and a lengthy process to develop a new chemical entitiy (NCE) with sufficient potency and selectivity to such target sequence. Antibody-based therapeutics on the other hand have significant potential as drugs because they have exquisite specificity to their target and a low inherent toxicity. In addition, the development time can be reduced considerably when compared to the development of new chemical entities (NCE's). However, conventional antibodies are difficult to raise against multimeric proteins where the receptor-binding domain of the ligand is a flexible loop as is the case with Interferon gamma (IFN-gamma). Heavy chain antibodies described in the invention which are derived from Camelidae, are known to be elicited against unexpected epitopes, such as the well-documented cavity-binding VHH's (WO97/49805; Lauwereys et al, EMBO J. 17, 5312, 1998)). Therefore, such heavy chain antibodies are inherently suited to bind to receptor binding domains of such ligands as Interferon gamma (IFN-gamma). In addition, such antibodies are known to be stable over long periods of time, therefore increasing their shelf-life (Perez et al, Biochemistry, 40, 74, 2001). Furthermore, such heavy chain antibody fragments (coined VHH) can be produced ‘en-masse’ in fermentors using cheap expression systems compared to mammalian cell culture fermentation, such as yeast or other microorganisms (EP 0 698 097).

The use of antibodies derived from sources such as mouse, sheep, goat, rabbit etc., and humanised derivatives thereof as a treatment for conditions which require a modulation of inflammation is problematic for several reasons. Traditional antibodies are not stable at room temperature, and have to be refrigerated for preparation and storage, requiring necessary refrigerated laboratory equipment, storage and transport, which contribute towards time and expense. Refrigeration is sometimes not feasible in developing countries. Furthermore, the manufacture or small-scale production of said antibodies is expensive because the mammalian cellular systems necessary for the expression of intact and active antibodies require high levels of support in terms of time and equipment, and yields are very low. Furthermore the large size of conventional antibodies would restrict tissue penetration, for example, at the site of inflamed tissue. Furthermore, traditional antibodies have a binding activity which depends upon pH, and hence are unsuitable for use in environments outside the usual physiological pH range such as, for example, in treating gastric bleeding, gastric surgery, inflammatory bowel disease, inflammation of the joint lining tissue (as in rheumatoid arthritis), destruction of the conducting fibers of the nervous tissue (as in multiple sclerosis). Furthermore, traditional antibodies are unstable at low or high pH and hence are not suitable for oral administration. However, it has been demonstrated that Camelidae antibodies resist harsh conditions, such as extreme pH, denaturing reagents and high temperatures (Ewert S et al, Biochemistry (2002) 41(11):3628-36), so making them suitable for delivery by oral administration. Furthermore, traditional antibodies have a binding activity which depends upon temperature, and hence are unsuitable for use in assays or kits performed at temperatures outside biologically active-temperature ranges (e.g. 37±20° C.).

Polypeptide therapeutics and in particular antibody-based therapeutics have significant potential as drugs because they have exquisite specificity to their target and a low inherent toxicity. However, it is known by the skilled addressee that an antibody which has been obtained for a therapeutically useful target requires additional modification in order to prepare it for human therapy, so as to avoid an unwanted immunological reaction in a human individual upon administration thereto. The modification process is commonly termed “humanisation”. It is known by the skilled artisan that antibodies raised in species, other than in humans, require humanisation to render the antibody therapeutically useful in humans. ((1) CDR grafting: Protein Design Labs: U.S. Pat. No. 6,180,370, U.S. Pat. No. 5,693,761; Genentech U.S. Pat. No. 6,054,297; Celltech: 460167, EP 626390, U.S. Pat. No. 5,859,205; (2) Veneering: Xoma: U.S. Pat. No. 5,869,619, U.S. Pat. No. 5,766,886, U.S. Pat. No. 5,821,123). There is a need for a method for producing antibodies which avoids the requirement for substantial humanisation, or which completely obviates the need for humanisation. There is a need for a new class of antibodies which have defined framework regions or amino acid residues and which can be administered to a human subject without the requirement for substantial humanisation, or the need for humanisation at all.

Another important drawback of conventional antibodies is that they are complex, large molecules and therefore relatively unstable, and they are sensitive to breakdown by proteases. This means that conventional antibody drugs cannot be administered orally, sublingually, topically, nasally, vaginally, rectally or by inhalation because they are not resistant to the low pH at these sites, the action of proteases at these sites and in the blood and/or because of their large size. They have to be administered by injection (intravenously, subcutaneously, etc.) to overcome some of these problems. Administration by injection requires specialist training in order to use a hypodermic syringe or needle correctly and safely. It further requires sterile equipment, a liquid formulation of the therapeutic polypeptide, vial packing of said polypeptide in a sterile and stable form and, of the subject, a suitable site for entry of the needle. Furthermore, subjects commonly experience physical and psychological stress prior to and upon receiving an injection. Therefore, there is need for a method for the delivery of therapeutic polypeptides which avoids the need for injection which is not only cost/time saving, but which would also be more convenient and more comfortable for the subject.

### AIMS OF THE INVENTION

It is an aim of the present invention is to provide polypeptides comprising one or more single domain antibodies which bind to Interferon gamma (IFN-gamma), homologues of said polypeptides, functional portions of homologues of said polypeptides. Said polypeptides modify the biological activity of IFN-gamma upon binding. Such polypeptides might bind into the receptor-binding domain of IFN-gamma, or might not bind in the receptor-binding domain.

It is a further aim of the present invention to provide single domain antibodies which may be any of the art, or any future single domain antibodies. Examples include, but are not limited to, heavy chain antibodies, antibodies naturally devoid of light chains, single domain antibodies derived from conventional 4-chain antibodies, engineered antibodies and single domain scaffolds other than those derived from antibodies. According to one aspect of the invention, a single domain antibody as used herein is a naturally occurring single domain antibody known as heavy chain antibody devoid of light chains (WO 9404678). For clarity reasons, this variable domain derived from a heavy chain antibody devoid of light chain will be called VHH or nanobody to distinguish it from the conventional VH of four chain immunoglobulins. Such a VHH molecule can be derived from antibodies raised in Camelidae species, for example in camel, dromedary, llama, alpaca and guanaco.

It is a further aim of the invention to provide a method of administering anti-IFN-gamma polypeptides intravenously, subcutaneously, orally, sublingually, topically, nasally, vaginally, rectally or by inhalation.

It is a further aim of the invention to enhance the binding affinity of monovalent single domain antibodies.

### SUMMARY OF THE INVENTION

One embodiment of the present invention is an anti-IFN-gamma polypeptide comprising at least one anti-IFN-gamma single domain antibody.

Another embodiment of the present invention is an anti-IFN-gamma polypeptide as described above, wherein at least one anti-IFN-gamma single domain antibody, is a Camelidae VHH antibody.

Another embodiment of the present invention is an anti-IFN-gamma polypeptide as described above wherein at least one single domain antibody corresponds to a sequence represented by any of SEQ ID NOs: 1 to 35

Another embodiment of the present invention is an anti-IFN-gamma polypeptide as described above further comprising at least one single domain antibody directed against a serum protein.

Another embodiment of the present invention is an anti-IFN-gamma polypeptide as described above wherein a serum protein is any of serum albumin, serum immunoglobulins, thyroxine-binding protein, transferring, or fibrinogen.

Another embodiment of the present invention is an anti-IFN-gamma polypeptide as described above wherein an anti-serum protein single domain antibody correspond to a sequence represented by any of SEQ ID NOs: 36 to 39 and 62 to 74.

Another embodiment of the present invention is an anti-IFN-gamma polypeptide as described above corresponding to a sequence represented by any of SEQ ID NOs: 40 to 42.

Another embodiment of the present invention is an anti-IFN-gamma polypeptide as described above further comprising at least one single domain antibody selected from the group consisting of anti-TNF-alpha single domain antibody, anti-TNF-alpha receptor single domain antibody and anti-IFN-gamma receptor single domain antibody.

Another embodiment of the present invention is an anti-IFN-gamma polypeptide as described above, wherein the number of single domain antibodies directed against IFN-gamma is at least two.

Another embodiment of the present invention is an anti-IFN-gamma polypeptide as described above corresponding to a sequence represented by any of SEQ ID NOs: 59 to 61.

Another embodiment of the present invention is an anti-IFN-gamma polypeptide as described above, wherein at least one single domain antibody is a humanized Camelidae VHHs.

Another embodiment of the present invention is a composition comprising an anti-IFN-gamma polypeptide as described above together with at least one single domain antibody from the group consisting of anti-TNF-alpha single domain antibody, anti-TNF-alpha receptor single domain antibody and anti-IFN-gamma receptor single domain antibody, for simultaneous, separate or sequential administration to a subject.

Another embodiment of the present invention is an anti-IFN-gamma polypeptide as described above, or a composition as described above wherein at least one anti-TNF-alpha single domain antibody correspond to a sequence represented by any of SEQ ID NOs: 43 to 58.

Another embodiment of the present invention is an anti-IFN-gamma polypeptide as described above, or a composition as described above, wherein said single domain antibody is an homologous sequence, a functional portion, or a functional portion of an homologous sequence of the full length single domain antibody.

Another embodiment of the present invention is an anti-IFN-gamma polypeptide as described above, or a composition as described above, wherein the anti-IFN-gamma polypeptide is an homologous sequence, a functional portion, or a functional portion of an homologous sequence of the full length anti-IFN-gamma polypeptide.

Another embodiment of the present invention is an anti-IFN-gamma polypeptide as described above, or a composition as described above wherein said single domain antibodies are Camelidae VHHs.

Another embodiment of the present invention is a nucleic acid encoding an anti-IFN-gamma polypeptide as described above.

Another embodiment of the present invention is a method of identifying an agent that modulates the binding of an anti-IFN-gamma polypeptide as described above, to IFN-gamma comprising the steps of:

(a) contacting an anti-IFN-gamma polypeptide as described above with a target that is IFN-gamma, in the presence and absence of a candidate modulator under conditions permitting binding between said polypeptide and target, and

(b) measuring the binding between the polypeptide and target of step (a), wherein a decrease in binding in the presence of said candidate modulator, relative to the binding in the absence of said candidate modulator identified said candidate modulator as an agent that modulates the binding of an anti-IFN-gamma polypeptide as described above and IFN-gamma.

Another embodiment of the present invention is a method of identifying an agent that modulates IFN-gamma-mediated disorders through the binding of an anti-IFN-gamma polypeptide as described above to IFN-gamma comprising:

(a) contacting an anti-IFN-gamma polypeptide as described above with a target that is IFN-gamma, in the presence and absence of a candidate modulator under conditions permitting binding between said polypeptide and target, and

(b) measuring the binding between the polypeptide and target of step (a), wherein a decrease in binding in the presence of said candidate modulator, relative to the binding in the absence of said candidate modulator identified, said candidate modulator as an agent that modulates IFN-gamma-mediated disorders.

Another embodiment of the present invention is a method of identifying an agent that modulates the binding of IFN-gamma to its receptor through the binding of an anti-IFN-gamma polypeptide as described above to IFN-gamma comprising:

(a) contacting an anti-IFN-gamma polypeptide as described above with a target that is IFN-gamma, in the presence and absence of a candidate modulator under conditions permitting binding between said polypeptide and target, and

(b) measuring the binding between the polypeptide and target of step (a), wherein a decrease in binding in the presence of said candidate modulator, relative to the binding in the absence of said candidate modulator identified said candidate modulator as an agent that modulates the binding of IFN-gamma to its receptor.

Another embodiment of the present invention is a kit for screening for agents that modulate IFN-gamma-mediated disorders comprising an anti-IFN-gamma polypeptide as described above and IFN-gamma.

Another embodiment of the present invention is an unknown agent that modulates the binding of an anti-IFN-gamma polypeptide as described above to IFN-gamma, identified according to the method as described above.

Another embodiment of the present invention is an unknown agent that modulates IFN-gamma-mediated disorders, identified according to the methods as described above.

Another embodiment of the present invention is an unknown agent as described above wherein said disorders are one or more of inflammation, rheumatoid arthritis, Crohn's disease, ulcerative colitis, inflammatory bowel syndrome and multiple sclerosis.

Another embodiment of the present invention is an anti-IFN-gamma polypeptide as described above, or a nucleic acid as described above, or a composition as described above, or an agent as described above for treating and/or preventing and/or alleviating disorders relating to inflammatory processes.

Another embodiment of the present invention is a use of an ant-IFN-gamma polypeptide as described above or a nucleic acid as described above, or a composition as described above, or an agent as described above for the preparation of a medicament for treating and/or preventing and/or alleviating disorders relating to inflammatory reactions.

Another embodiment of the present invention is an anti-IFN-gamma polypeptide as described above or a composition as described above, for treating and/or preventing and/or alleviating disorders requiring the delivery of a IFN-gamma modulating polypeptide that is able pass through the gastric environment without being inactivated.

Another embodiment of the present invention is a use of an anti-IFN-gamma polypeptide as described above or a composition as described above, for the preparation of a medicament for treating, preventing and/or alleviating the symptoms of disorders requiring the delivery of a IFN-gamma modulating polypeptide that is able pass through the gastric environment without being inactivated.

Another embodiment of the present invention is an anti-IFN-gamma polypeptide as described above or a composition as described above, for treating and/or preventing and/or alleviating disorders requiring the delivery of a IFN-gamma modulator to the vaginal and/or rectal tract.

Another embodiment of the present invention is a use of an anti-IFN-gamma polypeptide as described above or a composition as described above, for the preparation of a medicament for treating, preventing and/or alleviating the symptoms of disorders requiring the delivery of a IFN-gamma modulator to the vaginal and/or rectal tract.

Another embodiment of the present invention is an anti-IFN-gamma polypeptide as described above or a composition as described above, for treating and/or preventing and/or alleviating disorders requiring the delivery of a therapeutic compound to the upper respiratory tract and lung.

Another embodiment of the present invention is a use of an anti-IFN-gamma polypeptide as described above or a composition as described above, for the preparation of a medicament for treating, preventing and/or alleviating the symptoms of disorders requiring the delivery of a therapeutic compound to the upper respiratory tract and lung.

Another embodiment of the present invention is an anti-IFN-gamma polypeptide as described above or a composition as described above, for treating and/or preventing and/or alleviating disorders requiring the delivery of a IFN-gamma modulator, wherein said disorder increases the permeability of the intestinal mucosa.

Another embodiment of the present invention is a use of an anti-IFN-gamma polypeptide as described above or a composition as described above, for the preparation of a medicament for treating, preventing and/or alleviating the symptoms of disorders requiring the delivery of a IFN-gamma modulator, wherein said disorder increases the permeability of the intestinal mucosa.

Another embodiment of the present invention is an anti-IFN-gamma polypeptide as described above or a composition as described above, for treating and/or preventing and/or alleviating disorders requiring delivery of a IFN-gamma modulator that is able pass through the tissues beneath the tongue.

Another embodiment of the present invention is a use of an anti-IFN-gamma polypeptide as described above or a composition as described above, for the preparation of a medicament for treating, preventing and/or alleviating the symptoms of disorders requiring delivery of a IFN-gamma modulator that is able pass through the tissues beneath the tongue.

Another embodiment of the present invention is an anti-IFN-gamma polypeptide as described above or a composition as described above, for treating and/or preventing and/or alleviating disorders requiring delivery of a IFN-gamma modulator that is able pass through the skin.

Another embodiment of the present invention is a use of an anti-IFN-gamma polypeptide as described above or a composition as described above, for the preparation of a medicament for treating, preventing and/or alleviating the symptoms of disorders requiring delivery of a IFN-gamma modulator that is able pass through the skin.

Another embodiment of the present invention is a method as described above, a kit as described above, a nucleic acid or agent as described above, use of a nucleic acid or agent as described above, a composition as described above, use of a composition as described above, an anti-IFN-gamma polypeptide as described above, use of an anti-IFN-gamma polypeptide as described above wherein said disorders are any of inflammation, rheumatoid arthritis, Crohn's disease, ulcerative colitis, inflammatory bowel syndrome, multiple sclerosis, Addison's disease, Autoimmune hepatitis, Autoimmune parotitis, Diabetes Type I, Epididymitis, Glomerulonephritis, Graves' disease, Guillain-Barre syndrome, Hashimoto's disease, Hemolytic anemia, Systemic lupus erythematosus, Male infertility, Multiple sclerosis, Myasthenia Gravis, Pemphigus, Psoriasis, Rheumatic fever, Rheumatoid arthritis, Sarcoidosis, Scleroderma, Sjogren's syndrome, Spondyloarthropathies, Thyroiditis, and Vasculitis.

Another embodiment of the present invention is a composition comprising a nucleic acid or agent as described above, an anti-IFN-gamma polypeptide as described above, or a composition as described above, and a suitable pharmaceutical vehicle.

Another embodiment of the present invention is a method of diagnosing a disorder characterised by the dysfunction of IFN-gamma comprising:

(a) contacting a sample with an anti-IFN-gamma polypeptide as described above,

(b) detecting binding of said polypeptide to said sample, and

(c) comparing the binding detected in step (b) with a standard, wherein a difference in binding relative to said sample is diagnostic of a disorder characterised by dysfunction of IFN-gamma.

Another embodiment of the present invention is a kit for screening for a disorder cited above, using a method as described above.

Another embodiment of the present invention is a kit for screening for a disorder cited above comprising an isolated anti-IFN-gamma polypeptide as described above.

Another embodiment of the present invention is a use of an anti-IFN-gamma polypeptide as described above for the purification of said IFN-gamma.

Another embodiment of the present invention is a use of an anti-IFN-gamma polypeptide as described above for inhibiting the interaction between IFN-gamma and one or more IFN-gamma receptors.

Another embodiment of the present invention is a method for producing an anti-IFN-gamma polypeptide as described above comprising the steps of:

(a) obtaining double stranded DNA encoding a Camelidae VHH directed to IFN-gamma,

(b) cloning and expressing the DNA selected in step (b).

Another embodiment of the present invention is a method of producing an anti-IFN-gamma polypeptide as described above comprising:

(a) culturing host cells comprising nucleic acid capable of encoding an anti-IFN-gamma polypeptide as described above, under conditions allowing the expression of the polypeptide, and,

(b) recovering the produced polypeptide from the culture.

Another embodiment of the present invention is a method as described above, wherein said host cells are bacterial or yeast.

Another embodiment of the present invention is a kit for screening for any of inflammation, rheumatoid arthritis, Crohn's disease, ulcerative colitis, inflammatory bowel syndrome or multiple sclerosis comprising an anti-IFN-gamma polypeptide as described above.

### BRIEF DESCRIPTION OF FIGURES AND TABLES

FIG. 1 Specificity for human IFN-γ of the different libraries derived from llama 5 and 6

FIG. 2 Specificity for human IFN-γ of the pooled libraries derived from llama 22 and 23

FIG. 3 Specificity for human IFN-γ of the pooled libraries derived from llama 6

FIG. 4 Specificity for mouse IFN-γ of the pooled libraries derived from llama 29 and 31

FIG. 5 Binding of biotinylated human and mouse IFN-γ to neutravidine

FIG. 6 binding of biotinylated human and mouse IFN-γ to its receptor

FIG. 7 Representation of dose-dependent inhibition using a polyclonal anti-human IFN-γ antibody as described in example 10

FIG. 8 Capacity of clones selected in MP2 (experiment 1) to inhibit IFN-γ/receptor interaction as described in example 10

FIG. 9 Capacity of clones selected in MP3 (experiment 2) to inhibit IFN-γ/receptor interaction as described in example 10

FIG. 10 Capacity of clones selected in MP4 (experiment 3) to inhibit IFN-γ/receptor interaction as described in example 10

FIG. 11 Representation of the dose-dependent inhibition of MP3B4SRA and MP2F6SR as described in example 10

FIG. 12 Representation of the dose-dependent inhibition of MP3B4SRA and MP2F6SR as described in example 11

FIG. 13 Representation of the dose-dependent inhibition of monovalent and bivalent MP3B4SRA and MP2F6SR and bispecifc MP3B4SRA/MP2F6SR as described in example 13

FIG. 14 Representation of the dose-dependent inhibition of monovalent and bivalent MP3B4SRA and MP2F6SR and bispecifc MP3B4SRA/MP2F6SR as described in example 14

FIG. 15 Representation of dose-dependent inhibition of anti-mouse IFN-gamma VHHs as described in Example 10 and Table 5

Table 1 Overview of the libraries, their diversity and % insert derived from different llama's and tissues as described in example 1 and 2

Table 2 Overview of screening experiments of different selections for human IFN-γ specific VHH as described in example 6-1

Table 3 Overview of screening experiments of selections for mouse IFN-γ specific VHH as described in example 6-2

Table 4 Overview of amino acid sequence of human IFN-γ specific VHH's

Table 5 Overview of amino acid sequence of mouse IFN-γ specific VHH's

Table 6 Overview of IC50 of different IFN-γ specific VHH as described in example 10

Table 7 Overview of Anti-mouse serum albumin/anti-IFN-gamma

Table 8 Amino acid sequence listing of the peptides of aspects of present invention directed against TNF-alpha

Table 9 Amino acid sequence listing of the bi-valent and bi-specific peptides of aspects of present invention directed against IFN-gamma

Table 10 IC50 data of monovalent anti-IFN-gamma VHH's as described in Example 11.

Table 11 IC50 data of bi-valent and bi-specific anti-IFN-gamma VHH's an IgG/Fab derived from neutralizing polyclonal goat anti-human-IFN-gamma serum as described in Example 14.

Table 12 Fractional homologies between the amino acid sequences of anti-mouse serum albumin VHHs of the invention.

Table 13 Fractional homologies between anti-TNF-alpha VHHs of the invention.

Table 14 Percentage homologies between anti-IFN-gamma VHHs of the invention.

### DETAILED DESCRIPTION

The present invention relates to an anti-interferon gamma (IFN-gamma) polypeptide, comprising at least one single domain antibody directed against IFN-gamma. The invention also relates to nucleic acids capable of encoding said polypeptides.

Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. According to one aspect of the invention, a single domain antibody as used herein is a naturally occurring single domain antibody known as heavy chain antibody devoid of light chains. Such single domain antibodies are disclosed in WO 94/04678 for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, dromedary, alpaca and guanaco. Other species besides Camelidae may produce heavy chain antibodies naturally devoid of light chain; such VHHs are within the scope of the invention.

VHHs, according to the present invention, and as known to the skilled addressee are heavy chain variable domains derived from immunoglobulins naturally devoid of light chains such as those derived from Camelidae as described in WO 94/04678 (and referred to hereinafter as VHH domains or nanobodies). VHH molecules are about 10× smaller than IgG molecules. They are single polypeptides and very stable, resisting extreme pH and temperature conditions. Moreover, they are resistant to the action of proteases which is not the case for conventional antibodies. Furthermore, in vitro expression of VHHs produces high yield, properly folded functional VHHs. In addition, antibodies generated in Camelids will recognize epitopes other than those recognised by antibodies generated in vitro through the use of antibody libraries or via immunisation of mammals other than Camelids (WO 9749805). As such, anti-IFN-gamma VHH's may interact more efficiently with IFN-gamma than conventional antibodies, thereby blocking its interaction with the IFN-gamma receptor more efficiently.

According to the invention, IFN-gamma is derived from any species. Examples of species relevant to the invention include as rabbits, goats, mice, rats, cows, calves, camels, llamas, monkeys, donkeys, guinea pigs, chickens, sheep, dogs, cats, horses, and preferably humans.

IFN-gamma is also a fragment of IFN-gamma, capable of eliciting an immune response. IFN-gamma is also a fragment of IFN-gamma, capable of binding to a single domain antibody raised against the full length IFN-gamma.

A single domain antibody directed against IFN-gamma means single domain antibody that it is capable of binding to IFN-gamma with an affinity of better than 10⁻⁶ M.

One embodiment of the present invention is an anti-IFN-gamma polypeptide wherein the single domain antibody comprises Camelidae VHH directed against IFN-gamma.

Another embodiment of the present invention is an anti-IFN-gamma polypeptide, wherein a single domain antibody corresponds to a sequence represented by any of SEQ ID NOs: 1 to 29 as shown in Table 4. Said sequences are derived from Camelidae heavy chain antibodies (VHHs) which are directed against human IFN-gamma.

The present invention further relates to an anti-IFN-gamma polypeptide, wherein a single domain antibody is a VHH directed against IFN-gamma, wherein the VHH belongs to a class having human-like sequences. The class is characterised in that the VHHs carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45 according to the Kabat numbering. As such, peptides belonging to this class show a high amino acid sequence homology to human VH framework regions and said peptides might be administered to a human directly without expectation of an unwanted immune response therefrom, and without the burden of further humanisation.

A human-like class of Camelidae single domain antibodies represented by SEQ ID No. 24 and 27 have been described in WO03035694 and contain the hydrophobic FR2 residues typically found in conventional antibodies of human origin or from other species, but compensating this loss in hydrophilicity by the charged arginine residue at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation.

Therefore, one aspect of the present invention allows for the direct administration of an anti-IFN-gamma polypeptide, wherein the single domain antibodies belong to the humanized class of VHH, and comprise a sequence represented by any of SEQ ID NO: 24 or 27, to a patient in need of the same.

Any of the VHHs as used by the invention may be of the traditional class or of the classes of human-like Camelidae antibodies. Said antibodies may be directed against whole IFN-gamma or a fragment thereof, or a fragment of a homologous sequence thereof. These polypeptides include the full length Camelidae antibodies, namely Fc and VHH domains, chimeric versions of heavy chain Camelidae antibodies with a human Fc domain or VHH's by themselves or derived fragments.

Anti-serum albumin VHH's may interact in a more efficient way with serum albumin than conventional antibodies which is known to be a carrier protein. As a carrier protein some of the epitopes of serum albumin may be inaccessible by bound proteins, peptides and small chemical compounds. Since VHH's are known to bind into ‘unusual’ or non-conventional epitopes such as cavities (WO 97/49805), the affinity of such VHH's to circulating albumin may be increased.

The present invention also relates to the finding that an anti-IFN-gamma polypeptide as disclosed herein further comprising one or more single domain antibodies directed against one or more serum proteins of a subject surprisingly has significantly prolonged half-life in the circulation of said subject compared with the half-life of the anti-IFN-gamma polypeptide when not part of said construct. Examples of such anti-IFN-gamma polypeptides are represented in Table 7 by SEQ ID NOs: 40 to 42. Furthermore, the said anti-IFN-gamma polypeptides were found to exhibit the same favourable properties of VHHs such as high stability remaining intact in mice, extreme pH resistance, high temperature stability and high target affinity.

Another embodiment of the present invention is an anti-IFN-gamma polypeptide further comprising one or more single domain antibodies directed against one or more serum proteins, said anti-IFN-gamma polypeptide comprising a sequence corresponding to any represented by SEQ ID NOs: 40 to 42 (Table 7).

Another embodiment of the present invention is an anti-IFN-gamma polypeptide, wherein an anti-serum protein single domain antibody corresponds to a sequence represented by any of SEQ ID NOs: 36 to 39 and 62 to 74 as shown in Table 7

The serum protein may be any suitable protein found in the serum of subject, or fragment thereof. In one aspect of the invention, the serum protein is serum albumin, serum immunoglobulins, thyroxine-binding protein, transferrin, or fibrinogen. Depending on the intended use such as the required half-life for effective treatment and/or compartimentalisation of the target antigen, the VHH-partner can be directed to one of the above serum proteins.

Another aspect of the invention is an anti-IFN-gamma polypeptide as disclosed herein further comprising at least one polypeptide selected from the group consisting of an anti-TNF-alpha polypeptide, an anti-TNF-alpha receptor polypeptide and anti-IFN-gamma receptor polypeptide, such polypeptides joined to each other as described below

It is an embodiment of the invention that a single domain antibody directed against TNF-alpha corresponds to a sequence represented by any of SEQ ID NOs: 43 to 58 (Table 8).

One aspect of the invention is a method for treating autoimmune disease comprising administering to an individual an effective amount of an anti-IFN-gamma polypeptide further comprising at least one polypeptide selected from the group consisting of anti-TNF-alpha polypeptide, anti-IFN-gamma receptor polypeptide and anti-TNF-alpha receptor polypeptide, such polypeptides joined to each other as described below or given seperately.

Another embodiment of the invention is an anti-IFN-gamma polypeptide further comprising an anti-IFN-gamma receptor polypeptide for use in treating autoimmune diseases. The aforementioned bifunctional polypeptide may also be used to treat a subject wherein an antagonistic or blocking of the IFN-gamma receptor is required.

One aspect of the invention is a composition comprising an anti-IFN-gamma polypeptide as disclosed herein and at least one polypeptide selected from the group consisting of anti-TNF-alpha polypeptide, anti-TNF-alpha receptor polypeptide and anti-IFN-gamma receptor polypeptide, for simultaneous, separate or sequential administration to a subject.

One aspect of the invention is a method for treating autoimmune disease comprising administering to an individual an effective amount of an anti-IFN-gamma polypeptide and a least one polypeptide selected from the group consisting of anti-TNF-alpha polypeptide, anti-IFN-gamma receptor polypeptide and anti-TNF-alpha receptor polypeptide, simultaneously, separately or sequentially.

Another aspect of the invention is a kit containing an anti-IFN-gamma polypeptide and at least one polypeptide selected from the group consisting of anti-TNF-alpha polypeptide, anti-IFN-gamma receptor polypeptide and anti-TNF-alpha receptor polypeptide for simultaneous, separate or sequential administration to a subject. It is an aspect of the invention that the kit may be used according to the invention. It is an aspect of the invention that the kit may be used to treat the diseases as cited herein.

By simultaneous administration means the polypeptides are administered to a subject at the same time. For example, as a mixture of the polypeptides or a composition comprising said polypeptides. Examples include, but are not limited to a solution administered intraveneously, a tablet, liquid, topical cream, etc., wherein each preparation comprises the polypeptides of interest.

By separate administration means the polypeptides are administered to a subject at the same time or substantially the same time. The polypeptides are present in the kit as separate, unmixed preparations. For example, the different polypeptides may be present in the kit as individual tablets. The tablets may be administered to the subject by swallowing both tablets at the same time, or one tablet directly following the other.

By sequential administration means the polypeptides are administered to a subject sequentially. The polypeptides are present in the kit as separate, unmixed preparations. There is a time interval between doses. For example, one polypeptide might be administered up to 336, 312, 288, 264, 240, 216, 192, 168, 144, 120, 96, 72, 48, 24, 20, 16, 12, 8, 4, 2, 1, or 0.5 hours after the other component.

In sequential administration, one polypeptide may be administered once, or any number of times and in various doses before and/or after administration of another polypeptide. Sequential administration may be combined with simultaneous or sequential administration.

The medical uses of the anti-IFN-gamma polypeptide described below, also apply to the composition comprising an anti-IFN-gamma polypeptide as disclosed herein and at least one polypeptide selected from the group consisting of anti-TNF-alpha polypeptide, anti-TNF-alpha receptor polypeptide and anti-IFN-gamma receptor polypeptide, for simultaneous, separate or sequential administration to a subject as disclosed here above.

Another embodiment of the present invention is an anti-IFN-gamma polypeptide as disclosed herein, wherein the number of single domain antibodies directed against IFN-gamma is two or more. Such multivalent anti-IFN-gamma polypeptides as disclosed herein have the advantage of unusually high functional affinity for the target, displaying much higher than expected inhibitory properties compared to their monovalent counterparts.

Another embodiment of the present invention is an anti-IFN-gamma polypeptide wherein the number of single domain antibodies directed against IFN-gamma is two or more, said anti-IFN-gamma polypeptide comprises a sequence corresponding to any represented by SEQ ID NOs: 59 to 61 (Table 9).

The multivalent anti-IFN-gamma polypeptides have functional affinities that are several orders of magnitude higher than the monovalent parent anti-IFN-gamma polypeptides. The inventors have found that the functional affinities of these multivalent polypeptides are much higher than those reported in the prior art for bivalent and multivalent antibodies. Surprisingly, anti-IFN-gamma polypeptides of the present invention linked to each other directly or via a short linker sequence show much higher functional affinities than those found with multivalent conventional four-chain antibodies.

The inventors have found that such large increased functional activities can be detected preferably with antigens composed of multidomain and multimeric proteins, either in straight binding assays or in functional assays, e.g. cytotoxicity assays.

A multivalent anti-IFN-gamma polypeptide as used herein refers to a polypeptide comprising two or more anti-IFN-gamma polypeptides which have been covalently linked. The anti-IFN-gamma polypeptides may be identical in sequence or may be different in sequence, but are directed against the same target or antigen. Depending on the number of anti-IFN-gamma polypeptides linked, a multivalent anti-IFN-gamma polypeptide may be bivalent (2 anti-IFN-gamma polypeptides), trivalent (3 anti-IFN-gamma polypeptides), tetravalent (4 anti-IFN-gamma polypeptides) or have a higher valency molecules.

According to one aspect of the present invention, the anti-IFN-gamma polypeptides are linked to each other directly, without use of a linker. According to another aspect of the present invention, the anti-IFN-gamma polypeptides are linked to each other via a peptide linker sequence. Such linker sequence may be a naturally occurring sequence or a non-naturally occurring sequence. The linker sequence is expected to be non-immunogenic in the subject to which the anti-IFN-gamma polypeptides is administered. The linker sequence may provide sufficient flexibility to the multivalent anti-IFN-gamma polypeptide, at the same time being resistant to proteolytic degradation. A non-limiting example of a linker sequences is one that can be derived from the hinge region of VHHs described in WO 96/34103.

It is an aspect of the invention that the multivalent anti-IFN-gamma polypeptides disclosed above may be used instead of or as well as the single unit anti-IFN-gamma polypeptides in the above mentioned therapies and methods of delivery.

The single domain antibodies may be joined to form any of the polypeptides disclosed herein comprising more than one single domain antibody using methods known in the art or any future method. For example, they may be fused by chemical cross-linking by reacting amino acid residues with an organic derivatising agent such as described by Blattler et al, Biochemistry 24,1517-1524; EP294703. Alternatively, the single domain antibody may be fused genetically at the DNA level i.e. a polynucleotide construct formed which encodes the complete polypeptide construct comprising one or more anti-target single domain antibodies. A method for producing bivalent or multivalent VHH polypeptide constructs is disclosed in PCT patent application WO 96/34103. One way of VHH antibodies is via the genetic route by linking a VHH antibody coding sequences either directly or via a peptide linker. For example, the C-terminal end of the VHH antibody may be linked to the N-terminal end of the next single domain antibody. This linking mode can be extended in order to link additional single domain antibodies for the construction and production of tri-, tetra-, etc. functional constructs.

According to one aspect of the present invention, the single domain antibodies are linked to each other directly, without use of a linker. Contrary to joining bulky conventional antibodies where a linker sequence is needed to retain binding activity in the two subunits, polypeptides of the invention can be linked directly thereby avoiding potential problems of the linker sequence, such as antigenicity when administered to a human subject, instability of the linker sequence leading to dissociation of the subunits.

According to another aspect of the present invention, the single domain antibodies are linked to each other via a peptide linker sequence. Such linker sequence may be a naturally occurring sequence or a non-naturally occurring sequence. The linker sequence is expected to be non-immunogenic in the subject to which the anti-IFN-gamma polypeptide is administered. The linker sequence may provide sufficient flexibility to the anti-IFN-gamma polypeptide, at the same time being resistant to proteolytic degradation. A non-limiting example of a linker sequences is one that can be derived from the hinge region of VHHs described in WO 96/34103.

According to another aspect of the invention, multivalent single domain antibodies comprising more than two single domain antibodies can be linked to each other either directly or via a linker sequence. Such constructs are difficult to produce with conventional antibodies and due to steric hindrance of the bulky subunits, functionality will be lost or greatly diminished rather than increased considerably as seen with VHH's of the invention compared to the monovalent construct.

The polypeptide constructs disclosed herein may be made by the skilled artisan according to methods known in the art or any future method. For example, VHHs may be obtained using methods known in the art such as by immunising a camel and obtaining hybridomas therefrom, or by cloning a library of single domain antibodies using molecular biology techniques known in the art and subsequent selection by using phage display.

According to an aspect of the invention an anti-IFN-gamma polypeptide may be a homologous sequence of a full-length anti-IFN-gamma polypeptide. According to another aspect of the invention, an anti-IFN-gamma polypeptide may be a functional portion of a full-length anti-IFN-gamma polypeptide. According to another aspect of the invention, an anti-IFN-gamma polypeptide may be a homologous sequence of a full length anti-IFN-gamma polypeptide. According to another aspect of the invention, an anti-IFN-gamma polypeptide may be a functional portion of a homologous sequence of a full length anti-IFN-gamma polypeptide. According to an aspect of the invention an anti-IFN-gamma polypeptide may comprise a sequence of an anti-IFN-gamma polypeptide.

According to an aspect of the invention a single domain antibody used to form an anti-IFN-gamma polypeptide may be a complete single domain antibody (e.g. a VHH) or a homologous sequence thereof. According to another aspect of the invention, a single domain antibody used to form the anti-IFN-gamma polypeptide may be a functional portion of a complete single domain antibody. According to another aspect of the invention, a single domain antibody used to form the anti-IFN-gamma polypeptide may be a homologous sequence of a complete single domain antibody. According to another aspect of the invention, a single domain antibody used to form the anti-IFN-gamma polypeptide may be a functional portion of a homologous sequence of a complete single domain antibody.

As used herein, a homologous sequence of the present invention may comprise additions, deletions or substitutions of one or more amino acids, which do not substantially alter the functional characteristics of the polypeptides of the invention. The number of amino acid deletions or substitutions is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

A homologous sequence according to the present invention may be a sequence of an anti-IFN-gamma polypeptide modified by the addition, deletion or substitution of amino acids, said modification not substantially altering the functional characteristics compared with the unmodified polypeptide.

A homologous sequence of the present invention may be a polypeptide which has been humanised. The humanisation of antibodies of the new class of VHHs would further reduce the possibility of unwanted immunological reaction in a human individual upon administration.

A homologous sequence according to the present invention may be a sequence which exists in other Camelidae species such as, for example, camel, llama, dromedary, alpaca, guanaco etc.

Where homologous sequence indicates sequence identity, it means a sequence which presents a high sequence identity (more than 70%, 75%, 80%, 85%, 90%, 95% or 98% sequence identity) with the parent sequence and is preferably characterised by similar properties of the parent sequence, namely affinity, said identity calculated using known methods.

Alternatively, a homologous sequence may also be any amino acid sequence resulting from allowed substitutions at any number of positions of the parent sequence according to the formula below:

Ser substituted by Ser, Thr, Gly, and Asn;

Arg substituted by one of Arg, His, Gln, Lys, and Glu;

Leu substituted by one of Leu, Ile, Phe, Tyr, Met, and Val;

Pro substituted by one of Pro, Gly, Ala, and Thr;

Thr substituted by one of Thr, Pro, Ser, Ala, Gly, His, and Gln;

Ala substituted by one of Ala, Gly, Thr, and Pro;

Val substituted by one of Val, Met, Tyr, Phe, Ile, and Leu;

Gly substituted by one of Gly, Ala, Thr, Pro, and Ser;

Ile substituted by one of Ile, Met, Tyr, Phe, Val, and Leu;

Phe substituted by one of Phe, Trp, Met, Tyr, Ile, Val, and Leu;

Tyr substituted by one of Tyr, Trp, Met, Phe, Ile, Val, and Leu;

His substituted by one of His, Glu, Lys, Gin, Thr, and Arg;

Gln substituted by one of Gln, Glu, Lys, Asn, His, Thr, and Arg;

Asn substituted by one of Asn, Glu, Asp, Gln, and Ser;

Lys substituted by one of Lys, Glu, Gln, His, and Arg;

Asp substituted by one of Asp, Glu, and Asn;

Glu substituted by one of Glu, Asp, Lys, Asn, Gln, His, and Arg;

Met substituted by one of Met, Phe, Ile, Val, Leu, and Tyr.

A homologous nucleotide sequence according to the present invention may refer to nucleotide sequences of more than 50, 100, 200, 300, 400, 500, 600, 800 or 1000 nucleotides able to hybridize to the reverse-complement of the nucleotide sequence capable of encoding the parent sequence, under stringent hybridisation conditions (such as the ones described by Sambrook et al., Molecular Cloning, Laboratory Manuel, Cold Spring, Harbor Laboratory press, N.Y.).

As used herein, a functional portion refers to a sequence of a single domain antibody that is of sufficient size such that the interaction of interest is maintained with affinity of 1×10⁻⁶ M or better.

Alternatively, a functional portion comprises a partial deletion of the complete amino acid sequence and still maintains the binding site(s) and protein domain(s) necessary for the binding of and interaction with its target.

As used herein, a functional portion refers to less than 100% of the complete sequence (e.g., 99%, 90%, 80%, 70%, 60%, 50%, 40%, 30%, 20%, 10%, 5%, 1% etc.), but comprising 5 or more amino acids or 15 or more nucleotides.

Targets as mentioned herein such as TNF-alpha, TNF-alpha receptor, IFN-gamma receptor, serum proteins (e.g. serum albumin, serum immunoglobulins, thyroxine-binding protein, transferrin, fibrinogen) and IFN-gamma may be fragments of said targets. Thus a target is also a fragment of said target, capable of eliciting an immune response. A target is also a fragment of said target, capable of binding to a single domain antibody raised against the full length target.

A fragment as used herein refers to less than 100% of the sequence (e.g., 99%, 90%, 80%, 70%, 60%, 50%, 40%, 30%, 20%, 10% etc.), but comprising 5, 6, 7, 8, 9, 10, 12, 13, 14, 15, 16, 17, 18, 19, 20, 21, 22, 23, 24, 25 or more amino acids. A fragment is of sufficient length such that the interaction of interest is maintained with affinity of 1×10⁻⁶ M or better.

A fragment as used herein also refers to optional insertions, deletions and substitutions of one or more amino acids which do not substantially alter the ability of the target to bind to a single domain antibody raised against the wild-type target. The number of amino acid insertions deletions or substitutions is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

One embodiment of the present invention relates to a method for preparing modified polypeptides based upon llama antibodies by determining the amino acid residues of the antibody variable domain (VHH) which may be modified without diminishing the native affinity of the domain for antigen and while reducing its immunogenicity with respect to a heterologous species; the use of VHHs having modifications at the identified residues which are useful for administration to heterologous species; and to the VHH so modified.

More specifically, the invention relates to the preparation of modified VHHs, which are modified for administration to humans, the resulting VHH themselves, and the use of such “humanized” VHHs in the treatment of diseases in humans. By humanised is meant mutated so that immunogenicity upon administration in human patients is minor or nonexistent. Humanising a polypeptide, according to the present invention, comprises a step of replacing one or more of the Camelidae amino acids by their human counterpart as found in the human consensus sequence, without that polypeptide losing its typical character, i.e. the humanisation does not significantly affect the antigen binding capacity of the resulting polypeptide. Such methods are known by the skilled addressee. Humanization of Camelidae single domain antibodies requires the introduction and mutagenesis of a limited amount of amino acids in a single polypeptide chain. This is in contrast to humanization of scFv, Fab, (Fab)2 and IgG, which requires the introduction of amino acid changes in two chains, the light and the heavy chain and the preservation of the assembly of both chains.

Some VHH contain typical Camelidae hallmark residues at position 37, 44, 45 and 47 with hydrophilic characteristics. Replacement of the hydrophilic residues by human hydrophobic residues at positions 44 and 45 (E44G and R45L) did not have an effect on binding and/or inhibition. Further humanization may be required by substitution of residues in FR 1, such as position 1, 5, 28 and 30; FR3, such as positions 74, 75, 76, 83, 84, 93 and 94; and FR4, such as position 103, 104, 108 and 111 (all numbering according to the Kabat).

One embodiment of the present invention is a method for humanizing a VHH comprising the steps of replacing of any of the following residues either alone or in combination:
FR1 (position 1, 5, 28 and 30),
the hallmark amino acid at position 44 and 45 in FR2,
FR3 residues 74, 75, 76, 83, 84, 93 and 94,
and positions 103, 104, 108 and 111 in FR4;
   (numbering according to the Kabat numbering).

One embodiment of the present invention is an anti-IFN gamma polypeptide, or a nucleic acid capable of encoding said polypeptide for use in treating, preventing and/or alleviating the symptoms of disorders relating to inflammatory processes. IFN-gamma is involved in inflammatory processes, and the blocking of IFN-gamma action can have an anti-inflammatory effect, which is highly desirable in certain disease states such as, for example, Crohn's disease. Our Examples demonstrate VHH's according to the invention which bind IFN-gamma and moreover, block its binding to the IFN-gamma receptor.

The anti-IFN-gamma polypeptide of the present invention is applicable to autoimmune diseases, such as Addison's disease (adrenal), Autoimmune diseases of the ear (ear), Autoimmune diseases of the eye (eye), Autoimmune hepatitis (liver), Autoimmune parotitis (parotid glands), Crohn's disease (intestine), Diabetes Type I (pancreas), Epididymitis (epididymis), Glomerulonephritis (kidneys), Graves' disease (thyroid), Guillain-Barre syndrome (nerve cells), Hashimoto's disease (thyroid), Hemolytic anemia (red blood cells), Systemic lupus erythematosus (multiple tissues), Male infertility (sperm), Multiple sclerosis (nerve cells), Myasthenia Gravis (neuromuscular junction), Pemphigus (primarily skin), Psoriasis (skin), Rheumatic fever (heart and joints), Rheumatoid arthritis (joint lining), Sarcoidosis (multiple tissues and organs), Scleroderma (skin and connective tissues), Sjogren's syndrome (exocrine glands, and other tissues), Spondyloarthropathies (axial skeleton, and other tissues), Thyroiditis (thyroid), Vasculitis (blood vessels). Within parenthesis is the tissue affected by the disease. This listing of autoimmune diseases is intended to be exemplary rather than inclusive.

Autoimmune conditions for which the anti-IFN-gamma polypeptide of the present invention is applicable include, for example, AIDS, atopic allergy, bronchial asthma, eczema, leprosy, schizophrenia, inherited depression, transplantation of tissues and organs, chronic fatigue syndrome, Alzheimer's disease, Parkinson's disease, myocardial infarction, stroke, autism, epilepsy, Arthus's phenomenon, anaphylaxis, and alcohol and drug addiction. In the above-identified autoimmune conditions, the tissue affected is the primary target, in other cases it is the secondary target. These conditions are partly or mostly autoimmune syndromes. Therefore, in treating them, it is possible to use the same methods, or aspects of the same methods that are herein disclosed, sometimes in combination with other methods.

Another embodiment of the present invention is a use of an anti-IFN gamma polypeptide, or a nucleic acid capable of encoding said polypeptide for the preparation of a medicament for treating a disorder relating to inflammatory processes.

Examples of disorders further include rheumatoid arthritis, Crohn's disease, ulcerative colitis, inflammatory bowel syndrome and multiple sclerosis.

Polypeptides and nucleic acids according to the present invention may be administered to a subject by conventional routes, such as intravenously. However, a special property of the anti-IFN-gamma polypeptides of the invention is that they are sufficiently small to penetrate barriers such as tissue membranes and/or tumours and act locally thereon, and they are sufficiently stable to withstand extreme environments such as in the stomach. Therefore, another aspect of the present invention relates to the delivery of anti-IFN-gamma polypeptides.

A subject according to the invention can be any mammal susceptible to treatment by therapeutic polypeptides.

Oral delivery of anti-IFN-gamma polypeptides of the invention results in the provision of such molecules in an active form in the colon at local sites that are affected by the disorder. These sites may be highly inflamed and contain IFN-gamma-producing cells. The anti-IFN-gamma polypeptides of the invention which bind to IFN-gamma can neutralise the IFN-gamma locally, avoiding distribution throughout the whole body and thus limiting negative side-effects. Genetically modified microorganisms such as Micrococcus lactis are able to secrete antibody fragments. Such modified microorganisms can be used as vehicles for local production and delivery of antibody fragments in the intestine. By using a strain which produces a IFN-gamma neutralizing antibody fragment, inflammatory bowel syndrome could be treated.

Another aspect of the invention involves delivering anti-INF-gamma polypeptides as described herein by using surface expression on or secretion from non-invasive bacteria, such as Gram-positive host organisms like Lactococcus spec. using a vector such as described in WO 00/23471.

One embodiment of the present invention is an anti-IFN-gamma polypeptide as disclosed herein for use in treating, preventing and/or alleviating the symptoms of disorders susceptible to modulation by an IFN-gamma modulator that is able pass through the gastric environment without being inactivated.

Examples of disorders are any that cause inflammation, including but not limited to rheumatoid arthritis, Crohn's disease, ulcerative colitis, inflammatory bowel syndrome and multiple sclerosis. As known by persons skilled in the art, once in possession of said anti-IFN-gamma polypeptide, formulation technology may be applied to release a maximum amount of polypeptide in the right location (in the stomach, in the colon, etc.). This method of delivery is important for treating, prevent and/or alleviate the symptoms of disorder whose targets that are located in the gut system.

An aspect of the invention is a method for treating, preventing and/or alleviating the symptoms of a disorder susceptible to modulation by a therapeutic compound that is able pass through the gastric environment without being inactivated, by orally administering to a subject an anti-IFN-gamma polypeptide as disclosed herein.

Another embodiment of the present invention is a use of an anti-IFN-gamma polypeptide as disclosed herein for the preparation of a medicament for treating, preventing and/or alleviating the symptoms of disorders susceptible to modulaton by an IFN-gamma modulator that is able pass through the gastric environment without being inactivated.

An aspect of the invention is a method for delivering an IFN-gamma modulator to the gut system without being inactivated, by orally administering to a subject an anti-IFN-gamma polypeptide as disclosed herein.

An aspect of the invention is a method for delivering an IFN-gamma modulator to the bloodstream of a subject without being inactivated, by orally administering to a subject an anti-IFN-gamma polypeptide as disclosed herein.

Another embodiment of the present invention is an anti-IFN-gamma polypeptide as disclosed herein for use in treating, preventing and/or alleviating the symptoms of disorders susceptible to modulation by an IFN-gamma modulator delivered to the vaginal and/or rectal tract.

Examples of disorders are any that cause inflammation, including but not limited to rheumatoid arthritis, Crohn's disease, ulcerative colitis, inflammatory bowel syndrome and multiple sclerosis. In a non-limiting example, a formulation according to the invention comprises an anti-IFN-gamma polypeptide as disclosed herein comprising one or more VHHs directed against one or more targets in the form of a gel, cream, suppository, film, or in the form of a sponge or as a vaginal ring that slowly releases the active ingredient over time (such formulations are described in EP 707473, EP 684814, U.S. Pat. No. 5,629,001).

An aspect of the invention is a method for treating, preventing and/or alleviating the symptoms of disorders susceptible to modulation by a therapeutic compound to the vaginal and/or rectal tract, by vaginally and/or rectally administering to a subject an anti-IFN-gamma polypeptide as disclosed herein.

Another embodiment of the present invention is a use of an anti-IFN-gamma polypeptide as disclosed herein for the preparation of a medicament for treating, preventing and/or alleviating the symptoms of disorders susceptible to modulation by an IFN-gamma modulator delivered to the vaginal and/or rectal tract without being inactivated.

An aspect of the invention is a method for delivering an IFN-gamma modulator to the vaginal and/or rectal tract without being inactivated, by administering to the vaginal and/or rectal tract of a subject an anti-IFN-gamma polypeptide as disclosed herein.

An aspect of the invention is a method for delivering an IFN-gamma modulator to the bloodstream of a subject without being inactivated, by administering to the vaginal and/or rectal tract of a subject an anti-IFN-gamma polypeptide as disclosed herein.

Another embodiment of the present invention is an anti-IFN-gamma polypeptide as disclosed herein, for use in treating, preventing and/or alleviating the symptoms of disorders susceptible to modulation by an IFN-gamma modulator delivered to the nose, upper respiratory tract and/or lung.

Examples of disorders are any that cause inflammation, including but not limited to rheumatoid arthritis, Crohn's disease, ulcerative colitis, inflammatory bowel syndrome and multiple sclerosis. In a non-limiting example, a formulation according to the invention, comprises an anti-IFN-gamma polypeptide as disclosed herein in the form of a nasal spray (e.g. an aerosol) or inhaler. Since the construct is small, it can reach its target much more effectively than therapeutic IgG molecules.

An aspect of the invention is a method for treating, preventing and/or alleviating the symptoms of disorders susceptible to modulation by a IFN-gamma modulator delivered to the upper respiratory tract and lung, by administering to a subject an anti-IFN-gamma polypeptide as disclosed herein, by inhalation through the mouth or nose.

Another aspect of the invention is a dispersible VHH composition, in particular dry powder dispersible VHH compositions, such as those described in U.S. Pat. No. 6,514,496. These dry powder compositions comprise a plurality of discrete dry particles with an average particle size in the range of 0.4-10 mm. Such powders are capable of being readily dispersed in an inhalation device. VHH's are particularly suited for such composition as lyophilized material can be readily dissolved (in the lung subsequent to being inhaled) due to its high solubilisation capacity (Muyldermans, S., Reviews in Molecular Biotechnology, 74, 277-303, (2001)). Alternatively, such lyophilized VHH formulations can be reconstituted with a diluent to generate a stable reconstituted formulation suitable for subcutaneous administration. For example, anti-IgE antibody formulations (Example 1; U.S. Pat. No. 6,267,958, EP 841946) have been prepared which are useful for treating allergic asthma.

Another embodiment of the present invention is a use of an anti-IFN-gamma polypeptide as disclosed herein for the preparation of a medicament for treating, preventing and/or alleviating the symptoms of disorders susceptible to modulation by an IFN-gamma modulator delivered to the nose, upper respiratory tract and/or lung without being inactivated.

An aspect of the invention is a method for delivering an IFN-gamma modulator to the nose, upper respiratory tract and lung, by administering to the nose, upper respiratory tract and/or lung of a subject an anti-IFN-gamma polypeptide as disclosed herein.

An aspect of the invention is a method for delivering an IFN-gamma modulator to the nose, upper respiratory tract and/or lung without being inactivated, by administering to the nose, upper respiratory tract and/or lung of a subject an anti-IFN-gamma polypeptide as disclosed herein.

An aspect of the invention is a method for delivering an IFN-gamma modulator to the bloodstream of a subject without being inactivated by administering to the nose, upper respiratory tract and/or lung of a subject an anti-IFN-gamma polypeptide as disclosed herein.

One embodiment of the present invention is an anti-IFN-gamma polypeptide as disclosed herein as disclosed herein for use in treating, preventing and/or alleviating the symptoms of disorders susceptible to modulation by an IFN-gamma modulator delivered to the intestinal mucosa, wherein said disorder increases the permeability of the intestinal mucosa. Because of their small size, an anti-IFN-gamma polypeptides as disclosed herein can pass through the intestinal mucosa and reach the bloodstream more efficiently in subjects suffering from disorders which cause an increase in the permeability of the intestinal mucosa, for example Crohn's disease.

An aspect of the invention is a method for treating, preventing and/or alleviating the symptoms of disorders susceptible to modulation by an IFN-gamma modulator delivered to the intestinal mucosa, wherein said disorder increases the permeability of the intestinal mucosa, by orally administering to a subject an anti-IFN-gamma polypeptide as disclosed herein.

This process can be even further enhanced by an additional aspect of the present invention—the use of active transport carriers. In this aspect of the invention, VHH is fused to a carrier that enhances the transfer through the intestinal wall into the bloodstream. In a non-limiting example, this “carrier” is a second VHH which is fused to the therapeutic VHH. Such fusion constructs are made using methods known in the art. The “carrier” VHH binds specifically to a receptor on the intestinal wall which induces an active transfer through the wall.

Another embodiment of the present invention is a use of an anti-IFN-gamma polypeptide as disclosed herein for the preparation of a medicament for treating, preventing and/or alleviating the symptoms of disorders susceptible to modulation by an IFN-gamma modulator delivered to the intestinal mucosa, wherein said disorder increases the permeability of the intestinal mucosa.

An aspect of the invention is a method for delivering an IFN-gamma modulator to the intestinal mucosa without being inactivated, by administering orally to a subject an anti-IFN-gamma polypeptide as disclosed herein.

An aspect of the invention is a method for delivering an IFN-gamma modulator to the bloodstream of a subject without being inactivated, by administering orally to a subject an anti-IFN-gamma polypeptide as disclosed herein.

This process can be even further enhanced by an additional aspect of the present invention—the use of active transport carriers. In this aspect of the invention, an anti-IFN-gamma polypeptide as disclosed herein is fused to a carrier that enhances the transfer through the intestinal wall into the bloodstream. In a non-limiting example, this “carrier” is a VHH which is fused to said polypeptide. Such fusion constructs made using methods known in the art. The “carrier” VHH binds specifically to a receptor on the intestinal wall which induces an active transfer through the wall.

One embodiment of the present invention is an anti-IFN-gamma polypeptide as disclosed herein for use in treating, preventing and/or alleviating the symptoms of disorders susceptible to modulation by an IFN-gamma modulator that is able pass through the tissues beneath the tongue effectively. Examples of disorders are any that cause inflammation, including but not limited to rheumatoid arthritis, Crohn's disease, ulcerative colitis, inflammatory bowel syndrome and multiple sclerosis. A formulation of said anti-IFN-gamma polypeptide as disclosed herein, for example, a tablet, spray, drop is placed under the tongue and adsorbed through the mucus membranes into the capillary network under the tongue.

An aspect of the invention is a method for treating, preventing and/or alleviating the symptoms of disorders susceptible to modulation by an IFN-gamma modulator that is able pass through the tissues beneath the tongue effectively, by sublingually administering to a subject an anti-IFN-gamma polypeptide.

Another embodiment of the present invention is a use of an anti-IFN-gamma polypeptide as disclosed herein for the preparation of a medicament for treating, preventing and/or alleviating the symptoms of disorders susceptible to modulation by an IFN-gamma modulator that is able to pass through the tissues beneath the tongue.

An aspect of the invention is a method for delivering an IFN-gamma modulator to the tissues beneath the tongue without being inactivated, by administering orally to a subject an anti-IFN-gamma polypeptide as disclosed herein.

An aspect of the invention is a method for delivering an IFN-gamma modulator to the bloodstream of a subject without being inactivated, by administering orally to a subject an anti-IFN-gamma polypeptide as disclosed herein.

One embodiment of the present invention is an anti-IFN-gamma polypeptide as disclosed herein comprising at least one single domain antibody for use in treating, preventing and/or alleviating the symptoms of disorders susceptible to modulation by an IFN-gamma modulator that is able pass through the skin effectively. Examples of disorders are any that cause inflammation, including but not limited to rheumatoid arthritis, Crohn's disease, ulcerative colitis, inflammatory bowel syndrome, complications associated with corneal eye transplant and multiple sclerosis. A formulation of said anti-IFN-gamma polypeptide, for example, a cream, film, spray, drop, patch, is placed on the skin and passes through.

An aspect of the invention is a method for treating, preventing and/or alleviating the symptoms of disorders susceptible to modulation by a therapeutic compound that is able pass through the skin effectively, by topically administering to a subject an anti-IFN-gamma polypeptide as disclosed herein.

Another aspect of the invention is the use of an anti-IFN-gamma polypeptide as disclosed herein as a topical ophthalmic composition for the treatment of ocular disorder, such as allergic disorders, which method comprises the topical administration of an ophthalmic composition comprising anti-IFN-gamma polypeptide as disclosed herein, said construct further comprising one or more anti-IgE VHH.

Another embodiment of the present invention is a use of an anti-IFN-gamma polypeptide as disclosed herein as disclosed herein for the preparation of a medicament for treating, preventing and/or alleviating the symptoms of disorders susceptible to modulation by an IFN-gamma modulator that is able pass through the skin effectively.

An aspect of the invention is a method for delivering an IFN-gamma modulator to the skin without being inactivated, by administering topically to a subject an anti-IFN-gamma polypeptide as disclosed herein.

An aspect of the invention is a method for delivering an IFN-gamma modulator to the bloodstream of a subject, by administering topically to a subject an anti-IFN-gamma polypeptide as disclosed herein.

In another embodiment of the present invention, an anti-IFN-gamma polypeptide as disclosed herein further comprises a carrier single domain antibody (e.g. VHH) which acts as an active transport carrier for transport said anti-IFN-gamma polypeptide as disclosed herein, the lung lumen to the blood.

Examples of disorders are any that cause inflammation, including but not limited to rheumatoid arthritis, Crohn's disease, ulcerative colitis, inflammatory bowel syndrome and multiple sclerosis.

A anti-IFN-gamma polypeptide further comprising a carrier binds specifically to a receptor present on the mucosal surface (bronchial epithelial cells) resulting in the active transport of the polypeptide from the lung lumen to the blood. The carrier single domain antibody may be fused to the anti-IFN-gamma polypeptide. Such fusion constructs made using methods known in the art and are described herein. The “carrier” single domain antibody binds specifically to a receptor on the mucosal surface which induces an active transfer through the surface.

Another aspect of the present invention is a method to determine which single domain antibodies (e.g. VHHs) are actively transported into the bloodstream upon nasal administration. Similarly, a naive or immune VHH phage library can be administered nasally, and after different time points after administration, blood or organs can be isolated to rescue phages that have been actively transported to the bloodstream. A non-limiting example of a receptor for active transport from the lung lumen to the bloodstream is the Fc receptor N (FcRn). One aspect of the invention includes the VHH molecules identified by the method. Such VHH can then be used as a carrier VHH for the delivery of a therapeutic VHH to the corresponding target in the bloodstream upon nasal administration.

In one aspect of the invention, one can use an anti-IFN-gamma polypeptide as disclosed herein an homologous sequence thereof, a functional portion thereof or a functional portion thereof an homologous sequence thereof, in order to screen for agents that modulate the binding of the polypeptide to IFN-gamma. When identified in an assay that measures binding or said polypeptide displacement alone, agents will have to be subjected to functional testing to determine whether they would modulate the action of the antigen in vivo. Examples of screening assays are given below primarily in respect of SEQ ID NO: 3, though any anti-IFN-gamma polypeptide may be appropriate.

In an example of a displacement experiment, phage or cells expressing IFN-gamma or a fragment thereof are incubated in binding buffer with, for example, a polypeptide represented by SEQ ID NO: 3 which has been labeled, in the presence or absence of increasing concentrations of a candidate modulator. To validate and calibrate the assay, control competition reactions using increasing concentrations of said polypeptide and which is unlabeled, can be performed. After incubation, cells are washed extensively, and bound, labeled polypeptide is measured as appropriate for the given label (e.g., scintillation counting, fluorescence, etc.). A decrease of at least 10% in the amount of labeled polypeptide bound in the presence of candidate modulator indicates displacement of binding by the candidate modulator. Candidate modulators are considered to bind specifically in this or other assays described herein if they displace 50% of labeled polypeptide (sub-saturating polypeptide dose) at a concentration of 1 μM or less.

Alternatively, binding or displacement of binding can be monitored by surface plasmon resonance (SPR). Surface plasmon resonance assays can be used as a quantitative method to measure binding between two molecules by the change in mass near an immobilized sensor caused by the binding or loss of binding of, for example, the polypeptide represented by SEQ ID NO: 3 from the aqueous phase to IFN-gamma, or fragment thereof immobilized in a membrane on the sensor. This change in mass is measured as resonance units versus time after injection or removal of the said polypeptide or candidate modulator and is measured using a Biacore Biosensor (Biacore AB). IFN-gamma, or fragment thereof can be for example immobilized on a sensor chip (for example, research grade CM5 chip; Biacore AB) in a thin film lipid membrane according to methods described by Salamon et al. (Salamon et al., 1996, Biophys J. 71: 283-294; Salamon et al., 2001, Biophys. J. 80: 1557-1567; Salamon et al., 1999, Trends Biochem. Sci. 24: 213-219, each of which is incorporated herein by reference.). Sarrio et al. demonstrated that SPR can be used to detect ligand binding to the GPCR A(1) adenosine receptor immobilized in a lipid layer on the chip (Sarrio et al., 2000, Mol. Cell. Biol. 20: 5164-5174, incorporated herein by reference). Conditions for the binding of SEQ ID NO:3 to IFN-gamma, or fragment thereof in an SPR assay can be fine-tuned by one of skill in the art using the conditions reported by Sarrio et al. as a starting point.

SPR can assay for modulators of binding in at least two ways. First, a polypeptide represented by SEQ ID NO: 3, for example, can be pre-bound to immobilized IFN-gamma, or fragment thereof, followed by injection of candidate modulator at a concentration ranging from 0.1 nM to 1 μM. Displacement of the bound polypeptide can be quantitated, permitting detection of modulator binding. Alternatively, the membrane-bound IFN-gamma, or fragment thereof can be pre-incubated with a candidate modulator and challenged with, for example, a polypeptide represented by SEQ ID NO: 3. A difference in binding affinity between said polypeptide and IFN-gamma, or fragment thereof pre-incubated with the modulator, compared with that between said polypeptide and IFN-gamma, or fragment thereof in absence of the modulator will demonstrate binding or displacement of said polypeptide in the presence of modulator. In either assay, a decrease of 10% or more in the amount of said polypeptide bound in the presence of candidate modulator, relative to the amount of said polypeptide bound in the absence of candidate modulator indicates that the candidate modulator inhibits the interaction of IFN-gamma, or fragment thereof and said polypeptide.

Another method of detecting inhibition of binding of, for example, a polypeptide represented by SEQ ID NO: 3, to IFN-gamma, or fragment thereof uses fluorescence resonance energy transfer (FRET). FRET is a quantum mechanical phenomenon that occurs between a fluorescence donor (D) and a fluorescence acceptor (A) in close proximity to each other (usually <100 Å of separation) if the emission spectrum of D overlaps with the excitation spectrum of A. The molecules to be tested, e.g. a polypeptide represented by SEQ ID NO: 3 and a IFN-gamma, or fragment thereof, are labelled with a complementary pair of donor and acceptor fluorophores. While bound closely together by the IFN-gamma: polypeptide interaction, the fluorescence emitted upon excitation of the donor fluorophore will have a different wavelength from that emitted in response to that excitation wavelength when the said polypeptide and IFN-gamma, or fragment thereof are not bound, providing for quantitation of bound versus unbound molecules by measurement of emission intensity at each wavelength. Donor fluorophores with which to label the IFN-gamma, or fragment thereof are well known in the art. Of particular interest are variants of the A. Victoria GFP known as Cyan FP (CFP, Donor (D)) and Yellow FP (YFP, Acceptor (A)). As an example, the YFP variant can be made as a fusion protein with IFN-gamma, or fragment thereof. Vectors for the expression of GFP variants as fusions (Clontech) as well as flurophore-labeled reagents (Molecular Probes) are known in the art. The addition of a candidate modulator to the mixture of fluorescently-labelled polypeptide and YFP-IFN-gamma will result in an inhibition of energy transfer evidenced by, for example, a decrease in YFP fluorescence relative to a sample without the candidate modulator. In an assay using FRET for the detection of IFN-gamma:polypeptide interaction, a 10% or greater decrease in the intensity of fluorescent emission at the acceptor wavelength in samples containing a candidate modulator, relative to samples without the candidate modulator, indicates that the candidate modulator inhibits the IFN-gamma:polypeptide interaction.

A sample as used herein may be any biological sample containing IFN-gamma such as clinical (e.g. cell fractions, whole blood, plasma, serum, tissue, cells, etc.), derived from clinical, agricultural, forensic, research, or other possible samples. The clinical samples may be from human or animal origin. The sample analysed can be both solid or liquid in nature. It is evident when solid materials are used, these are first dissolved in a suitable solution.

A variation on FRET uses fluorescence quenching to monitor molecular interactions. One molecule in the interacting pair can be labelled with a fluorophore, and the other with a molecule that quenches the fluorescence of the fluorophore when brought into close apposition with it. A change in fluorescence upon excitation is indicative of a change in the association of the molecules tagged with the fluorophore:quencher pair. Generally, an increase in fluorescence of the labelled IFN-gamma, or fragment thereof is indicative that anti-IFN-gamma polypeptide bearing the quencher has been displaced. For quenching assays, a 10% or greater increase in the intensity of fluorescent emission in samples containing a candidate modulator, relative to samples without the candidate modulator, indicates that the candidate modulator inhibits IFN-gamma: anti-IFN-gamma polypeptide interaction.

In addition to the surface plasmon resonance and FRET methods, fluorescence polarization measurement is useful to quantitate binding. The fluorescence polarization value for a fluorescently-tagged molecule depends on the rotational correlation time or tumbling rate. Complexes, such as those formed by IFN-gamma, or fragment thereof associating with a fluorescently labelled anti-IFN-gamma polypeptide, have higher polarization values than uncomplexed, labelled polypeptide. The inclusion of a candidate inhibitor of the IFN-gamma:anti-IFN-gamma polypeptide interaction results in a decrease in fluorescence polarization, relative to a mixture without the candidate inhibitor, if the candidate inhibitor disrupts or inhibits the interaction of IFN-gamma, or fragment thereof with said polypeptide. Fluorescence polarization is well suited for the identification of small molecules that disrupt the formation of IFN-gamma:anti-IFN-gamma polypeptide complexes. A decrease of 10% or more in fluorescence polarization in samples containing a candidate modulator, relative to fluorescence polarization in a sample lacking the candidate modulator, indicates that the candidate modulator inhibits the IFN-gamma:anti-IFN-gamma polypeptide interaction.

Another alternative for monitoring IFN-gamma:anti-IFN-gamma polypeptide interactions uses a biosensor assay. ICS biosensors have been described in the art (Australian Membrane Biotechnology Research Institute; Cornell B, Braach-Maksvytis V, King L, Osman P, Raguse B, Wieczorek L, and Pace R. “A biosensor that uses ion-channel switches” Nature 1997, 387, 580). In this technology, the association of IFN-gamma, or fragment thereof and a anti-IFN-gamma polypeptide is coupled to the closing of gramacidin-facilitated ion channels in suspended membrane bilayers and thus to a measurable change in the admittance (similar to impedence) of the biosensor. This approach is linear over six orders of magnitude of admittance change and is ideally suited for large scale, high throughput screening of small molecule combinatorial libraries. A 10% or greater change (increase or decrease) in admittance in a sample containing a candidate modulator, relative to the admittance of a sample lacking the candidate modulator, indicates that the candidate modulator inhibits the interaction of IFN-gamma, or fragment thereof and said polypeptide. It is important to note that in assays testing the interaction of IFN-gamma, or fragment thereof with an anti-IFN-gamma polypeptide, it is possible that a modulator of the interaction need not necessarily interact directly with the domain(s) of the proteins that physically interact with said polypeptide. It is also possible that a modulator will interact at a location removed from the site of interaction and cause, for example, a conformational change in the IFN-gamma. Modulators (inhibitors or agonists) that act in this manner are nonetheless of interest as agents to modulate the binding of IFN-gamma to its receptor.

Any of the binding assays described can be used to determine the presence of an agent in a sample, e.g., a tissue sample, that binds to IFN-gamma, or fragment thereof, or that affects the binding of, for example, a polypeptide represented by SEQ ID NO: 3 to the IFN-gamma, or fragment thereof. To do so a IFN-gamma, or fragment thereof is reacted with said polypeptide in the presence or absence of the sample, and polypeptide binding is measured as appropriate for the binding assay being used. A decrease of 10% or more in the binding of said polypeptide indicates that the sample contains an agent that modulates the binding of said polypeptide to the IFN-gamma, or fragment thereof. Of course, the above-generalized method might easily be applied to screening for candidate modulators which alter the binding between any anti-IFN-gamma polypeptide of the invention, an homologous sequence thereof, a functional portion thereof or a functional portion of an homologous sequence thereof, and IFN-gamma or a fragment thereof.

One embodiment of the present invention is an unknown agent identified by the method disclosed herein.

One embodiment of the present invention is an unknown agent identified by the method disclosed herein for use in treating, preventing and/or alleviating the symptoms of disorders relating to inflammatory processes.

Another embodiment of the present invention is a use of an unknown agent identified by the method disclosed herein for use in treating, preventing and/or alleviating the symptoms of disorders relating to inflammatory processes.

Examples of disorders include rheumatoid arthritis, Crohn's disease, ulcerative colitis, inflammatory bowel syndrome and multiple sclerosis

A cell that is useful according to the invention is preferably selected from the group consisting of bacterial cells such as, for example, E. coli, yeast cells such as, for example, S. cerevisiae, P. pastoris, insect cells or mammal cells.

A cell that is useful according to the invention can be any cell into which a nucleic acid sequence encoding a polypeptide comprising an anti-IFN-gamma of the invention, an homologous sequence thereof, a functional portion thereof or a functional portion of an homologous sequence thereof according to the invention can be introduced such that the polypeptide is expressed at natural levels or above natural levels, as defined herein. Preferably a polypeptide of the invention that is expressed in a cell exhibits normal or near normal pharmacology, as defined herein. Most preferably a polypeptide of the invention that is expressed in a cell comprises the nucleotide sequence capable of encoding any one of the amino acid sequences presented in Table 4 and 5 or capable of encoding an amino acid sequence that is at least 70% identical to the amino acid sequence presented in Table 4 and 5.

According to a preferred embodiment of the present invention, a cell is selected from the group consisting of COS7-cells, a CHO cell, a LM (TK-) cell, a NIH-3T3 cell, HEK-293 cell, K-562 cell or a 1321N1 astrocytoma cell but also other transfectable cell lines.

In general, “therapeutically effective amount”, “therapeutically effective dose” and “effective amount” means the amount needed to achieve the desired result or results (modulating IFN-gamma binding; treating or preventing inflammation). One of ordinary skill in the art will recognize that the potency and, therefore, an “effective amount” can vary for the various compounds that modulate IFN-gamma binding used in the invention. One skilled in the art can readily assess the potency of the compound.

As used herein, the term “compound” refers to an anti-IFN-gamma polypeptide or a composition of the present invention, or a nucleic acid capable of encoding said polypeptide (or composition) or an agent identified according to the screening method described herein, or said polypeptides comprising one or more derivatised amino acids.

By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained.

The anti-IFN polypeptides of the present invention are useful for treating or preventing conditions in a subject and comprises administering a pharmaceutically effective amount of a compound or composition.

The anti-IFN polypeptides of the present invention are useful for treating or preventing conditions relating to rheumatoid arthritis, Crohn's disease, ulcerative colitis, inflammatory bowel syndrome and multiple sclerosis in a subject and comprises administering a pharmaceutically effective amount of a compound or composition that binds IFN-gamma.

The anti-IFN-gamma polypeptides as disclosed here in are useful for treating or preventing conditions in a subject and comprises administering a pharmaceutically effective amount of a compound combination with another, such as, for example, aspirin.

The anti-IFN-gamma polypeptides as disclosed here in are useful for treating or preventing conditions relating to rheumatoid arthritis, Crohn's disease, ulcerative colitis, inflammatory bowel syndrome and multiple sclerosis in a subject and comprises administering a pharmaceutically effective amount of a compound combination with another, such as, for example, aspirin.

The present invention is not limited to the administration of formulations comprising a single compound of the invention. It is within the scope of the invention to provide combination treatments wherein a formulation is administered to a patient in need thereof that comprises more than one compound of the invention.

Conditions mediated by IFN-gamma include, but are not limited rheumatoid arthritis, Crohn's disease, ulcerative colitis, inflammatory bowel syndrome and multiple sclerosis.

A compound useful in the present invention can be formulated as pharmaceutical compositions and administered to a mammalian host, such as a human patient or a domestic animal in a variety of forms adapted to the chosen route of administration, i.e. but not limited to, orally or parenterally, intranassally by inhalation, intravenous, intramuscular, topical or subcutaneous routes.

A compound of the present invention can also be administered using gene therapy methods of delivery. See, e.g., U.S. Pat. No. 5,399,346, which is incorporated by reference in its entirety. Using a gene therapy method of delivery, primary cells transfected with the gene for the compound of the present invention can additionally be transfected with tissue specific promoters to target specific organs, tissue, grafts, tumors, or cells.

Thus, the present compound may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage.

The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compound may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, hydroxyalkyls or glycols or water-alcohol/glycol blends, in which the present compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compound to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compound can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician. Also the dosage of the compound varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen could include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

The invention provides for an agent that is a modulator of IFN-gamma/IFN-gamma-receptor interactions.

The candidate agent may be a synthetic agent, or a mixture of agents, or may be a natural product (e.g. a plant extract or culture supernatant). A candidate agent according to the invention includes a small molecule that can be synthesized, a natural extract, peptides, proteins, carbohydrates, lipids etc.

Candidate modulator agents from large libraries of synthetic or natural agents can be screened. Numerous means are currently used for random and directed synthesis of saccharide, peptide, and nucleic acid based agents. Synthetic agent libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are available and can be prepared. Altematively, libraries of natural agents in the form of bacterial, fungal, plant and animal extracts are available from e.g., Pan Laboratories (Bothell, Wash.) or MycoSearch (NC), or are readily producible by methods well known in the art. Additionally, natural and synthetically produced libraries and agents are readily modified through conventional chemical, physical, and biochemical means.

Useful agents may be found within numerous chemical classes. Useful agents may be organic agents, or small organic agents. Small organic agents have a molecular weight of more than 50 yet less than about 2,500 daltons, preferably less than about 750, more preferably less than about 350 daltons. Exemplary classes include heterocycles, peptides, saccharides, steroids, and the like. The agents may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like.

For primary screening, a useful concentration of a candidate agent according to the invention is from about 10 mM to about 100 μM or more (i.e. 1 mM, 10 mM, 100 mM, 1 M etc.). The primary screening concentration will be used as an upper limit, along with nine additional concentrations, wherein the additional concentrations are determined by reducing the primary screening concentration at half-log intervals (e.g. for 9 more concentrations) for secondary screens or for generating concentration curves.

A high throughput screening kit according to the invention comprises all the necessary means and media for performing the detection of an agent that modulates IFN-gamma/IFN-gamma receptor interactions by interacting with IFN-gamma, or fragment thereof in the presence of a polypeptide, preferably at a concentration in the range of 1 μM to 1 mM.

The kit comprises the following. Recombinant cells of the invention, comprising and expressing the nucleotide sequence encoding IFN-gamma, or fragment thereof, which are grown according to the kit on a solid support, such as a microtiter plate, more preferably a 96 well microtiter plate, according to methods well known to the person skilled in the art especially as described in WO 00/02045. Alternatively IFN-gamma, or fragment thereof is supplied in a purified form to be immobilized on, for example, a 96 well microtiter plate by the person skilled in the art. Alternatively IFN-gamma, or fragment thereof is supplied in the kit pre-immobilized on, for example, a 96 well microtiter plate. The IFN-gamma may be whole IFN-gamma or a fragment thereof.

Modulator agents according to the invention, at concentrations from about 1 μM to 1 mM or more, are added to defined wells in the presence of an appropriate concentration of anti-IFN-gamma polypeptide, an homologous sequence thereof, a functional portion thereof or a functional portion of an homologous sequence thereof, said concentration of said polypeptide preferably in the range of 1 μM to 1 mM. Kits may contain one or more anti-IFN-gamma polypeptide (e.g. one or more of a polypeptide represented by any of the SEQ ID NOs: 1 to 29 or other anti-IFN-gamma polypeptides, an homologous sequence thereof, a functional portion thereof or a functional portion of an homologous sequence thereof).

Binding assays are performed as according to the methods already disclosed herein and the results are compared to the baseline level of, for example IFN-gamma, or fragment thereof binding to an anti-IFN-gamma polypeptide, an homologous sequence thereof, a functional portion thereof or a functional portion of an homologous sequence thereof, but in the absence of added modulator agent. Wells showing at least 2 fold, preferably 5 fold, more preferably 10 fold and most preferably a 100 fold or more increase or decrease in IFN-gamma-polypeptide binding (for example) as compared to the level of activity in the absence of modulator, are selected for further analysis.

The invention provides for kits useful for screening for modulators of IFN-gamma/IFN-gamma receptor binding, as well as kits useful for diagnosis of disorders characterised by dysfunction of IFN-gamma. The invention also provides for kits useful for screening for modulators of disorders as well as kits for their diagnosis, said disorders characterised by one or more process involving IFN-gamma. Kits useful according to the invention can include an isolated IFN-gamma, or fragment thereof. Alternatively, or in addition, a kit can comprise cells transformed to express IFN-gamma, or fragment thereof. In a further embodiment, a kit according to the invention can comprise a polynucleotide encoding IFN-gamma, or fragment thereof. In a still further embodiment, a kit according to the invention may comprise the specific primers useful for amplification of IFN-gamma, or fragment thereof. Kits useful according to the invention can comprise an isolated IFN-gamma polypeptide, a homologue thereof, or a functional portion thereof. A kit according to the invention can comprise cells transformed to express said polypeptide. Kits may contain more than one polypeptide. In a further embodiment, a kit according to the invention can comprise a polynucleotide encoding IFN-gamma, or fragment thereof. In a still further embodiment, a kit according to the invention may comprise the specific primers useful for amplification of a macromolecule such as, for example, IFN-gamma, or a fragment thereof. All kits according to the invention will comprise the stated items or combinations of items and packaging materials therefore. Kits will also include instructions for use.

### EXAMPLES

The invention is illustrated by the following non-limiting examples.

### Example 1

### Immunization

Four llama's (llama 5, 6, 22 and 23) were immunized intramuscularly with human IFN-gamma (PeproTech Inc, USA, Cat Nr: 300-02) using an appropriate animal-friendly adjuvant Stimune (Cedi Diagnostics BV, The Netherlands). Two llama's (llama 29 and 31) were immunized intramuscularly with mouse IFN-gamma (Protein Expression & Purification core facility, VIB-RUG, Belgium) using an appropriate animal-friendly adjuvant Stimune (Cedi Diagnostics BV, The Netherlands). The llama's received 6 injections at weekly intervals, the first two injections containing each 100 μg of IFN-gamma, the last four injections containing each 50 μg of IFN-gamma. Four days after the last immunization a blood sample (PBL1) of 150 ml and a lymph node biopsy (LN) was collected from each animal and sera were prepared. Ten days after the last immunization a second blood sample (PBL2) of 150 ml was taken from each animal and sera were prepared. Peripheral blood lymphocytes (PBLs), as the genetic source of the llama heavy chain immunoglobulins (HcAbs), were isolated from the blood sample using a Ficoll-Paque gradient (Amersham Biosciences) yielding 5×10⁸ PBLs. The maximal diversity of antibodies is expected to be equal to the number of sampled B-lymphocytes, which is about 10% of the number of PBLs (5×10⁷). The fraction of heavy-chain antibodies in llama is up to 20% of the number of B-lymphocytes. Therefore, the maximal diversity of HcAbs in the 150 ml blood sample is calculated as 10⁷ different molecules. Total RNA was isolated from PBLs and lymph nodes according to the method of Chomczynski and Sacchi (1987).

### Example 2

### Repertoire Cloning

cDNA was prepared on 200 μg total RNA with MMLV Reverse Transcriptase (Gibco BRL) using oligo d(T) oligonucleotides (de Haard et al., 1999). The cDNA was purified with a phenol/chloroform extraction, followed by an ethanol precipitation and subsequently used as template to amplify the VHH repertoire.

In a first PCR, the repertoire of both conventional (1.6 kb) and heavy-chain (1.3 kb) antibody gene segments were amplified using a leader specific primer (5′-GGCTGAGCTCGGTGGTCCTGGCT-3′) and the oligo d(T) primer (5′-AACTGGAAGAATTCGCGGCCGCAGGAATTTTTTTTTTTTTTTTTT-3′). The resulting DNA fragments were separated by agarose gel electrophoresis and the 1.3 kb fragment encoding heavy-chain antibody segments was purified from the agarose gel. A second PCR was performed using a mixture of FR1 reverse primers (WO03/054016 sequences ABL037 to ABL043) and the same oligo d(T) forward primer.

The PCR products were digested with SfiI (introduced in the FR1 primer) and BstElI (naturally occurring in framework 4). Following gel electrophoresis, the DNA fragments of approximately 400 basepairs were purified from gel and ligated into the corresponding restriction sites of phagemid pAX004 to obtain a library of cloned VHHs after electroporation of Escherichia coli TG1. pAX004 allows the production of phage particles, expressing the individual VHHs as a fusion protein with a c-myc tag, a hexahistidine tag and the geneIII product. The diversity obtained after electroporation of TG1 cells is presented in Table 1. The percentage insert was determined in PCR using a combination of vector based primers.

### Example 3

### Rescue of the Library and Phage Preparation

The library was grown at 37° C. in 10 ml 2×TY medium containing 2% glucose, and 100 μg/ml ampicillin, until the OD₆₀₀ₙₘ reached 0.5. M13KO7 phages (10¹²) were added and the mixture was incubated at 37° C. for 2×30 minutes, first without shaking, then with shaking at 100 rpm. Cells were centrifuged for 5 minutes at 4,500 rpm at room temperature. The bacterial pellet was resuspended in 50 ml of 2×TY medium containing 100 μg/ml ampicillin and 25 μg/ml kanamycin, and incubated overnight at 37° C. with vigorously shaking at 250 rpm. The overnight cultures were centrifuged for 15 minutes at 4,500 rpm at 4° C. Phages were PEG precipitated (20% poly-ethylene-glycol and 1.5 M NaCl) for 30 minutes on ice and centrifuged for 20 minutes at 4,500 rpm. The pellet was resuspended in 1 ml PBS. Phages were again PEG precipitated for 10 minutes on ice and centrifuged for 10 minutes at 14,000 rpm and 4° C. The pellet was dissolved in 1 ml PBS-0.1% casein.

### Example 4

### Library Evaluation

The library was evaluated in a phage ELISA to examine whether the cloned repertoire contained significant IFN-γ specific VHH's. The repertoire was expressed on phage following infection with M13K07 helper phages as described in example 3.

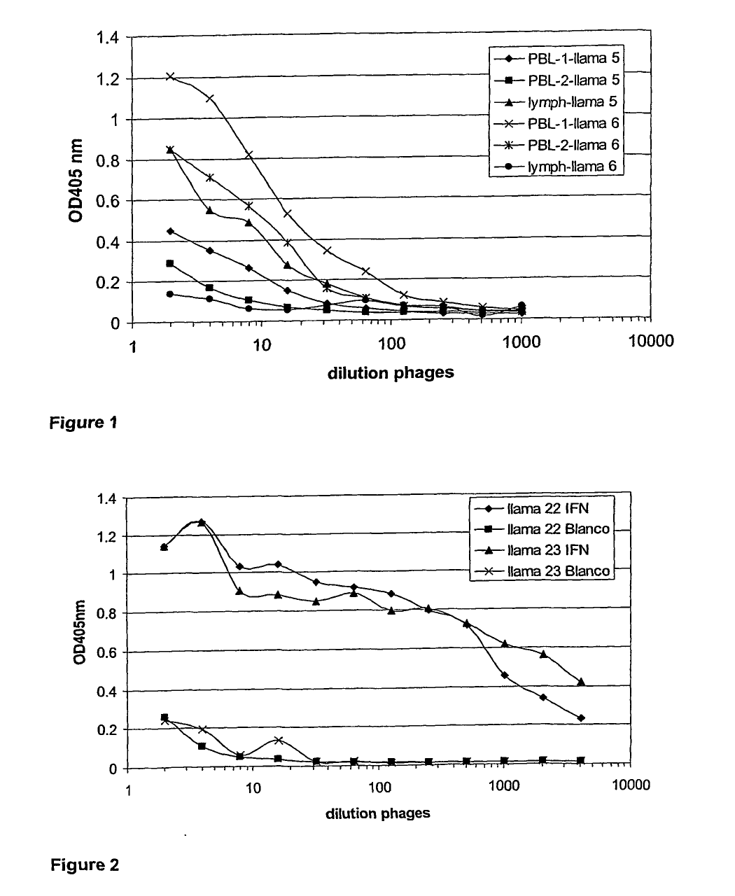

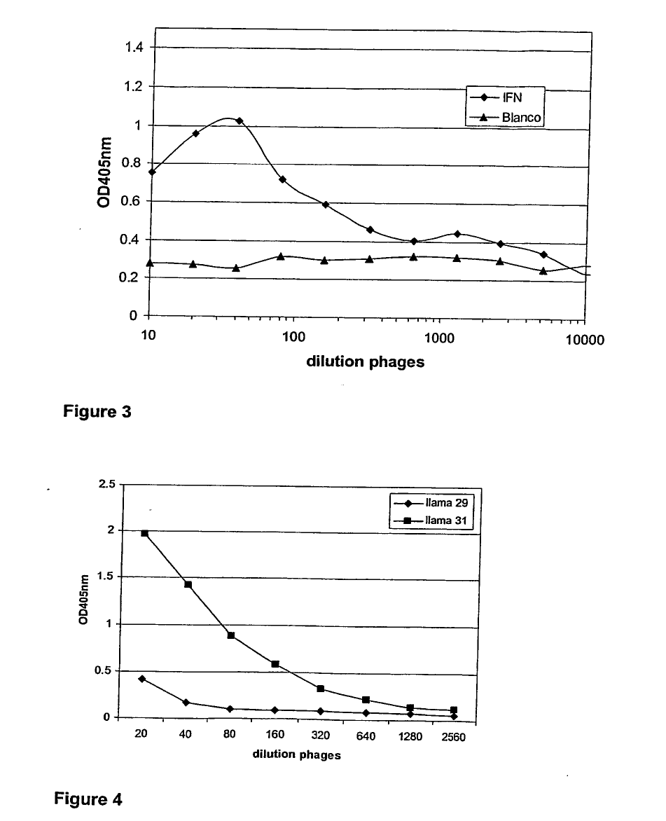

Human IFN-γ was solid phase coated at a concentration of 1 μg/ml overnight at 4° C. in a 96-well microtiterplate. Plates were washed 5 times with PBS/0.05% Tween-20. Plates were blocked using PBS+1% Caseine. A dilution serie of purified phages were added to the wells and incubated for 2 hrs at room temperature. Plates were washed 5 times with PBS/0.05% Tween-20. Bound phages were detected using the anti-M13 gene VIII-HRP conjugated monoclonal antibody (Amersham Biosciences) and ABTS/H₂O₂ as substrate. Plates were read at 405 nm after 30 minutes incubation at room temperature. The results of the phage ELISA are presented in FIGS. 1, 2 and 3.

To evaluate the mouse IFN-γ specific libraries, 96-well microtiter plates were coated with neutravidine at a concentration of 2 μg/well overnight at 4° C. Plates were washed 5 times with PBS/0.05% Tween-20. Wells were blocked with PBS+1% Caseine for 2 hrs at room temperature. Biotinylated mouse IFN-γ (see example 5) at a concentration of 1 μg/ml was captured overnight at 4° C. Plates were washed 5 times with PBS/0.05% Tween-20. A dilution serie of purified phages were added to the wells. Plates were washed 5 times with PBS/0.05% Tween-20. Bound phages were detected using the anti-M13 gene VIII-HRP conjugated monoclonal antibody (Amersham Biosciences). Plates were read at 405 nm after 30 minutes incubation at room temperature.

The results of the phage ELISA are presented in FIG. 4.

### Example 5

### Biotinylation of IFN-γ

100 μg human IFN-γ and 50 μg mouse IFN-γ was biotinylated using a 10-fold molar excess of biotinamidocaproic acid 3-sulfo N-hydroxysuccinimide ester (Sigma, Cat Nr. B1022). Biotinylation was performed in 50 mM Na₂CO₃ pH=8 and reaction was stopped after 2 hrs incubation at room temperature using 10 mM Tris-HCl pH=7.5. Free biotine was removed using dialysis. Biotinylation was validated by binding of biotinylated IFN-γ to neutravidine and to IFN-γ receptor.

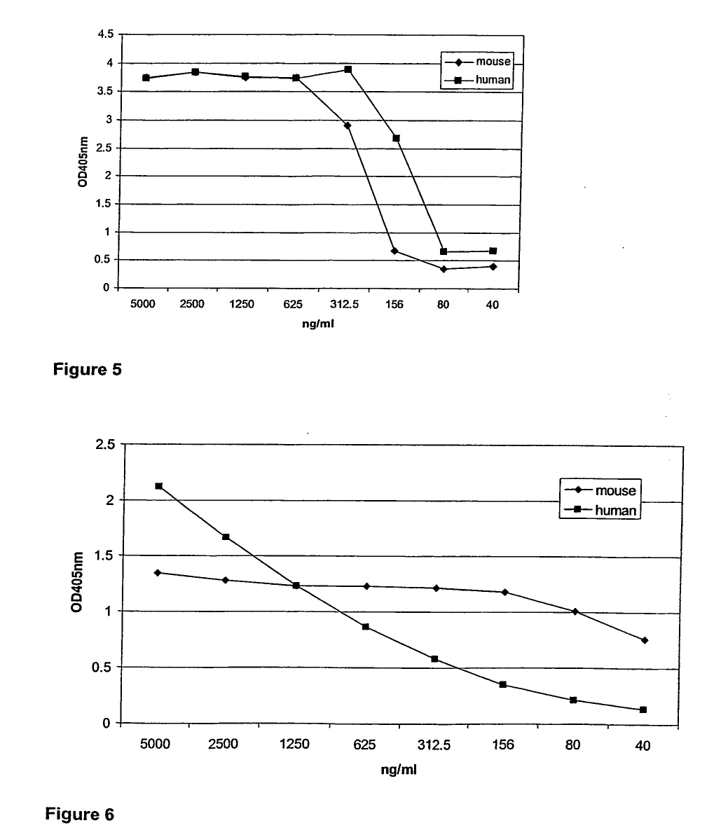

96-well microtiter plates were coated with 2 μg/ml neutravidine overnight at 4° C. Plates were washed 5 times with PBS/0.05% Tween-20. Plates were blocked for 2 hrs at room temperature with PBS+1% Caseine. A dilution serie of biotinylated human or mouse IFN-γ was incubated in the wells for 1 hr at room temperature. Plates were washed 5 times with PBS/0.05% Tween-20. Binding was detected using Extravidin-AP and pNPP. Plates were read at 405 nm after 30 minutes incubation at room temperature. Results are presented in FIG. 5.

96-well microtiter plates were coated with human IFN-γ receptor (IFN-γ R1 (R&D Systems, Cat Nr: 673-IR/CF) or mouse IFN-γ receptor (IFN-γ R1/Fc (R&D Systems, Cat Nr:1026-GR) at 1 μg/ml in PBS overnight at 4° C. Plates were washed 5 times with PBS/0.05% Tween-20. Plates were blocked for 2 hrs at room temperature using PBS+1% Caseine. A dilution serie of biotinylated human or mouse IFN-γ was incubated for 1 hr at room temperature. Plates were washed 5 times with PBS/0.05% Tween-20. Binding was detected using Extravidin-AP and pNPP. Plates were read at 405 nm after 30 minutes incubation at room temperature. Results are presented in FIG. 6.

### Example 6-1

### Selection of Human IFN-γ Specific VHH

Phages were rescued and prepared as described above in example 3

Two approaches were followed to obtain IFN-γ specific binders:
a. Solid Phase Coated IFN-γ
Microtiter wells were coated with human IFN-γ at different concentrations of 10-0.4 μg/well overnight at 4° C. Plates were washed 5 times with PBS/0.05% Tween-20. Wells were blocked with PBS+1% caseine for 2 hrs at room temperature. Phages were incubated for 2 hrs at room temperature. Wells were washed 20 times with PBS+0.05% Tween-20. The two final washes were performed using PBS. Specific phages were eluted using 1 to 2 μg of IFN-γ R1 (R&D Systems, Cat Nr: 673-IR/CF) for 1 hr. As negative control elutions were performed using 10 μg Ovalbumine (Sigma, A2512) as irrelevant protein. Log phase growing TG1 cells were infected with the eluted phages and plated on selective medium. Enrichment was determined by the number of transfected TG1 colonies after selection using the receptor for elution as compared with negative control using ovalbumine for elution. Bacteria from selections showing enrichment were scraped and used for a second round of selection.
The bacteria were superinfected with helperphage to produce recombinant phages as described in example 3. Microtiter wells were coated with IFN-γ at different concentrations of 2-0.1 μg/well overnight at 4° C. Plates were washed 5 times with PBS/0.05% Tween-20. Wells were blocked with PBS+1% caseine for 2 hrs at room temperature. Phages were incubated for 2 hrs at room temperature. Wells were washed 20 times with PBS+0.05% Tween-20. The two final washes were performed using PBS. Specific phages were eluted using 1 to 2 μg of IFN-γ R1 or 10 μg Ovalbumine as irrelevant protein for 1 hr, subsequently overnight at 4° C. and subsequently, phages were eluted using 0.1 M glycine pH 2.5 for 15 minutes at room temperature and neutralized with 1M Tris-HCl pH=7.5. Log phase growing TG1 cells were infected with the eluted and neutralized phages and plated on selective medium. Enrichment was determined by the number of transfected TG1 colonies after selection using the receptor for elution as compared with negative control using ovalbumine for elution.
b. Biotinylated IFN-γ
Microtiter wells were coated with neutravidine at a concentration of 2 μg/ml overnight at 4° C. Plates were washed 5 times with PBS/0.05% Tween-20. Wells were blocked with PBS+1% caseine for 2 hrs at room temperature. Biotinylated human IFN-γ at a concentration of 100-10 ng/well was captured overnight at 4° C. Plates were washed 5 times with PBS/0.05% Tween-20. Phages were incubated for 2 hrs at room temperature. Wells were washed 20 times with PBS+0.05% Tween-20. The two final washes were performed using PBS. Specific phages were eluted using 1 to 2 μg of IFN-γ R1 (R&D Systems, Cat Nr: 673-IR/CF) for 1 hr. As negative control elutions were performed using 10 μg Ovalbumine (Sigma, A2512) as irrelevant protein. Log phase growing TG1 cells were infected with the eluted phages and plated on selective medium. Enrichment was determined by the number of transfected TG1 colonies after selection using the receptor for elution as compared with negative control using ovalbumine for elution. Bacteria from selections showing enrichment were scraped and used for a second round of selection.

Bacteria were superinfected with helperphage to produce recombinant phages. Microtiter wells were coated with neutravidine at a concentration of 2 μg/ml overnight at 4° C. Plates were washed 5 times with PBS/0.05% Tween-20. Wells were blocked with PBS+1% caseine for 2 hrs at room temperature. Biotinylated human IFN-γ at a concentration of 20-2.5 ng/100 μl was captured overnight at 4° C. Plates were washed 5 times with PBS/0.05% Tween-20. Phages were incubated for 2 hrs at room temperature. Wells were washed 20 times with PBS+0.05% Tween-20. The two final washes were performed using PBS. Specific phages were eluted using 1 to 2 μg of IFN-γ R1 or 10 μg Ovalbumine as irrelevant protein for 1 hr, subsequently overnight at 4° C. and subsequently, phages were eluted using 0.1 M glycine pH 2.5 for 15 minutes at room temperature and neutralized with 1M Tris-HCl pH=7.5. Log phase growing TG1 cells were infected with the eluted and neutralized phages and plated on selective medium. Enrichment was determined by the number of transfected TG1 colonies after selection using the receptor for elution as compared with negative control using ovalbumine for elution.

### Example 6-2

### Selection of Mouse IFN-γ Specific VHH

Phages were rescued and prepared as described above in example 3. Microtiter wells were coated with neutravidine at a concentration of 2 μg/ml overnight at 4° C. Plates were washed 5 times with PBS/0.05% Tween-20. Wells were blocked with PBS+1% caseine for 2 hrs at room temperature. Biotinylated mouse IFN-γ at a concentration of 200-30 ng/well was captured overnight at 4° C. Plates were washed 5 times with PBS/0.05% Tween-20. Phages were incubated for 2 hrs at room temperature. Wells were washed 20 times with PBS+0.05% Tween-20. The two final washes were performed using PBS. Specific phages were eluted using 1 μg of IFN-γ R1/Fc (R&D Systems, Cat Nr:1026-GR) for 1 hr. As negative control elutions were performed using 10 μg Ovalbumine (Sigma, A2512) as irrelevant protein. Log phase growing TG1 cells were infected with the eluted phages and plated on selective medium. Enrichment was determined by the number of transfected TG1 colonies after selection using the receptor for elution as compared with negative control using ovalbumine for elution. Bacteria from selections showing some enrichment were scraped and used for a second round of selection.

Bacteria were superinfected with helperphage to produce recombinant phages. Microtiter wells were coated with neutravidine at a concentration of 2 μg/ml overnight at 4° C. Plates were washed 5 times with PBS/0.05% Tween-20. Wells were blocked with PBS+1% caseine for 2 hrs at room temperature. Biotinylated mouse IFN-γ at a concentration of 30-2.5 ng/well was captured overnight at 4° C. Plates were washed 5 times with PBS/0.05% Tween-20. Phages were incubated for 2 hrs at room temperature. Wells were washed with PBS+0.05% Tween-20. The two final washes were performed using PBS. Specific phages were eluted using 1 to 2 μg of IFN-γ R1/Fc or 10 μg Ovalbumine as irrelevant protein for 1 hr, subsequently overnight at 4° C. and subsequently, phages were eluted using 0.1 M glycine pH 2.5 for 15 minutes at room temperature and neutralized with 1M Tris-HCl pH=7.5. Log phase growing TG1 cells were infected with the eluted and neutralized phages and plated on selective medium. Enrichment was determined by the number of transfected TG1 colonies after selection using the receptor for elution as compared with negative control using ovalbumine for elution.

### Example 7

### Specificity of Selected VHH's

Individual clones were picked, grown in 150 μl 2×TY containing 0.1% glucose and 100 μg/ml ampicillin in a microtiter plate at 37° C. until OD_{600 nm}=0.6. 1 mM IPTG and 5 mM MgSO₄ was added and the culture was incubated overnight at 37° C. ELISA was performed on the supernatant of the cultures to examine specificity of the selected clones. To examine the clones selected using solid phase coated human IFN-γ, plates were coated with human IFN-γ at a concentration of 5-10 μg/ml overnight at 4° C. Plates were washed 5 times with PBS/0.05% Tween-20. Wells were blocked with 1% caseine for 2 hrs at room temperature. Culture supernatant (⅓ diluted) was applied to the wells. Plates were washed 5 times with PBS/0.05% Tween-20. Detection was performed using anti-c-myc antibody, followed by anti-mouse-HRP and ABTS/H₂O₂ as substrate. Plates were read at 405 nm after 30 minutes incubation at room temperature.

To examine the clones selected using biotinylated human or mouse IFN-γ, wells were coated with neutravidine at a concentration of 2 μg/ml overnight at 4° C. Plates were washed 5 times with PBS/0.05% Tween-20. Wells were blocked with 1% caseine for 2 hrs at room temperature. Biotinylated mouse or human IFN-γ at a concentrabon of 1 μg/ml was captured overnight at 4° C. Plates were washed 5 times with PBS/0.05% Tween-20. Culture supernatant (⅓ diluted) was applied to the wells. Detection was performed using anti-c-myc antibody, followed by anti-mouse-HRP and ABTS/H₂O₂ as substrate. Plates were read at 405 nm after 30 minutes incubation at room temperature.

Results on binders against human IFN-γ are presented in Table 2. Results on binders against mouse IFN-γ are presented in Table 3.

### Example 8

### Diversity of Selected VHH's

PCR was performed using M13 reverse and genIII forward primers. The clones were analyzed using Hinf1 fingerprinting and representative clones were sequenced. Sequence analysis was performed resulting in the sequences and sequence families presented in Table 4 for human IFN-γ and in Table 5 for mouse IFN-γ.

### Example 9

### Expression and Purification of VHH

Small scale expressions were started after transformation of DNA into WK6 Escherichia coli cells.

Clones were grown in 50 ml 2×TY containing 0.1% glucose and 100 μg/ml ampicillin in a shaking flask at 37° C. until OD_{600 nm}=2.1 mM IPTG and 5 mM MgSO₄ was added and the culture was incubated for 3 more hours at 37° C. Cultures were centrifuged for 10 minutes at 4,500 rpm at 4° C. The pellet was frozen overnight at −20° C. Next, the pellet was thawed at room temperature for 40 minutes, re-suspended in 1 ml PBS/1 mM EDTA/1 M NaCl and shaken on ice for 1 hour. Periplasmic fraction was isolated by centrifugation for 10 minutes at 4° C. at 4,500 rpm. The supernatant containing the VHH was loaded on TALON (Clontech) and purified to homogeneity. The yield of VHH was calculated according to the extinction coefficient.

### Example 10

### Functional Characterization of Selected VHH's: Inhibition of Binding of IFN-γ to the IFN-γ Receptor by a VHH in an In-House Receptor-Binding Assay

VHH were expressed and purified as described in example 9. Binding was still observed when the periplasmic fractions were tested in an ELISA as described in example 7 (data not shown).

Purified. VHH was analyzed for the ability to inhibit human or mouse IFN-γ/IFN-γ receptor interaction.

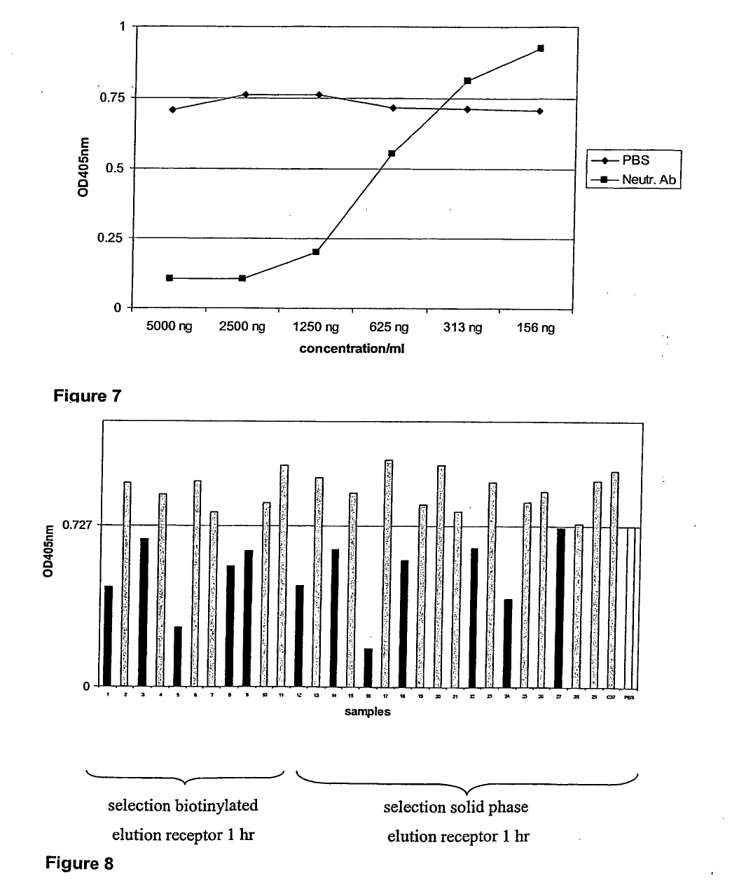

Mouse or human IFN-γ receptor was coated at a concentration of 1-2 μg/ml overnight at 4° C. Plates were washed 5 times with PBS/0.05% Tween-20. Wells were blocked with 1% caseine overnight at 4° C. VHH was pre-incubated with 20 ng biotinylated human or mouse IFN-γ for 30 minutes at room temperature. The mixture was applied to the wells and incubated for 1 hr at room temperature. Detection was performed using Extravidin-AP and pNPP as substrate. Plates were read at 405 nm after 30 minutes incubation at room temperature. Abcam AB 7812 polyclonal antibody was used as a positive control showing a dosis-dependent inhibition of human IFN-γ/IFN-γ receptor as presented in FIG. 7.

11 VHH molecules from experiment 1 (MP2 selection experiment) showed inhibition of human IFN-γ/IFN-γ receptor interaction. An irrelant VHH directed against Von Willebrand factor was included as negative control. The clones were selected using either solid phase coated or biotinylated human IFN-γ. FIG. 8 represents the MP2 selection.

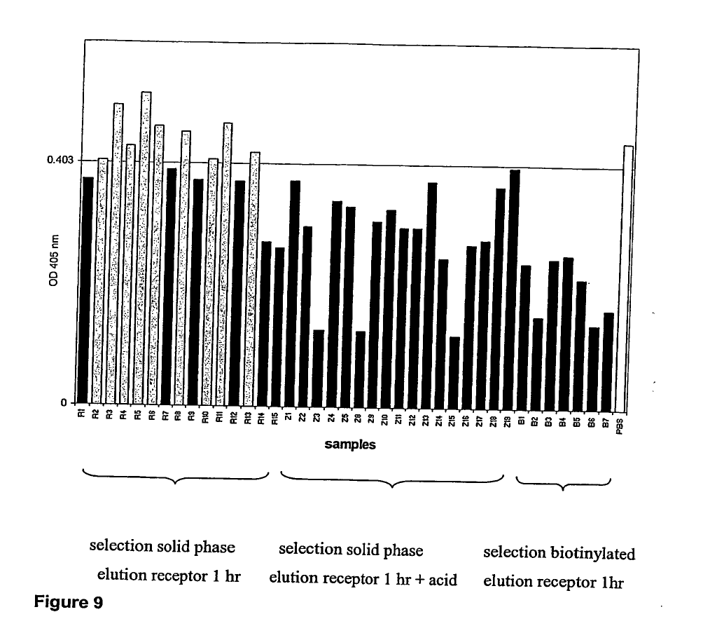

31 clones from experiment 2 (MP3 selection experiment) showed inhibition of human IFN-γ/IFN-γ receptor interaction. The clones were selected using either solid phase coated or biotinylated human IFN-γ and using different elution procedures. FIG. 9 represents the MP3 selection.

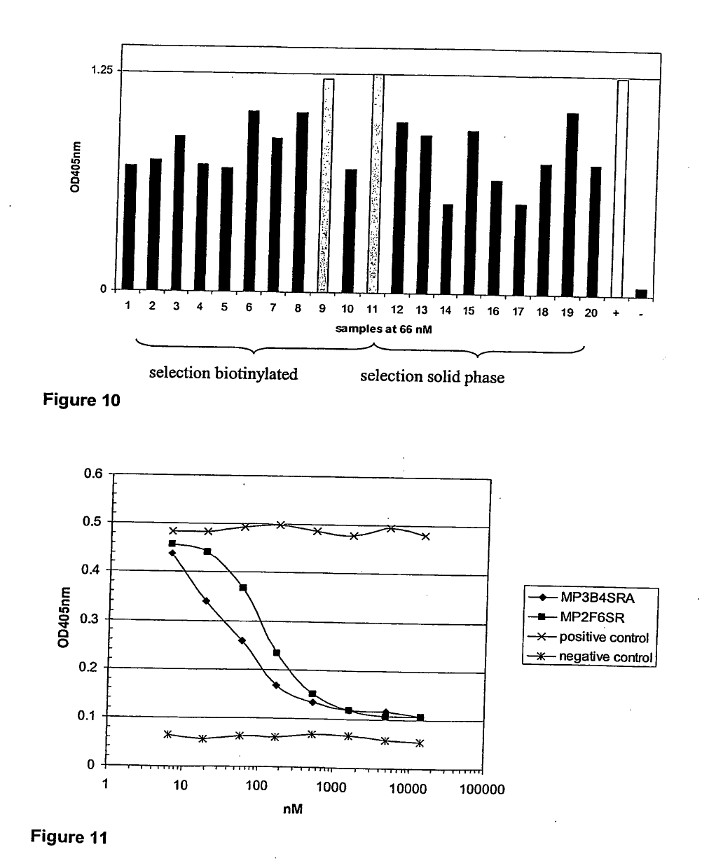

20 clones from experiment 3 (MP4 selection experiment) showed inhibition of human IFN-γ/IFN-γ receptor interaction. The clones were selected using either solid phase coated or biotinylated human IFN-γ. FIG. 10 represents the MP4 selection.

As presented in Table 6, a dose-dependent inhibition assay to determine the IC50 was performed for representative clones of each sequence family. The IC50 was defined as the concentration of VHH that inhibits the binding of IFN-γ to its receptor by 50%. From that experiment MP2 F6 SR and MP3 B4 SRA were identified as most potent inhibitors showing a good dose-responsiveness. A comparison of both VHH's is given in FIG. 11.

6 clones directed against mouse IFN-γ were analyzed for their capacity to inhibit mouse IFN-γ/IFN-γ receptor interaction. FIG. 15 represents the results.

### Example 11

### Functional Characterization of Selected VHH's: Inhibition of Binding of Human IFN-γ to the Human IFN-γ Receptor by a VHH in an in vitro Cell-Based Inhibition Assay

Purified VHH were tested in cytotoxicity assays. Endotoxin was depleted from the samples using Tx-114. The samples were incubated for 30 minutes with 0.2% Tx-114. Subsequently, the mixture was incubated at 37° C. for 30 minutes and centrifuged for 10 minutes at 14,000 rpm. The upper phase was harvested and treated once more. There was no difference in binding in ELISA (example 7) or inhibition capacity (example 10) between Tx-114 treated and untreated VHH (data not shown).

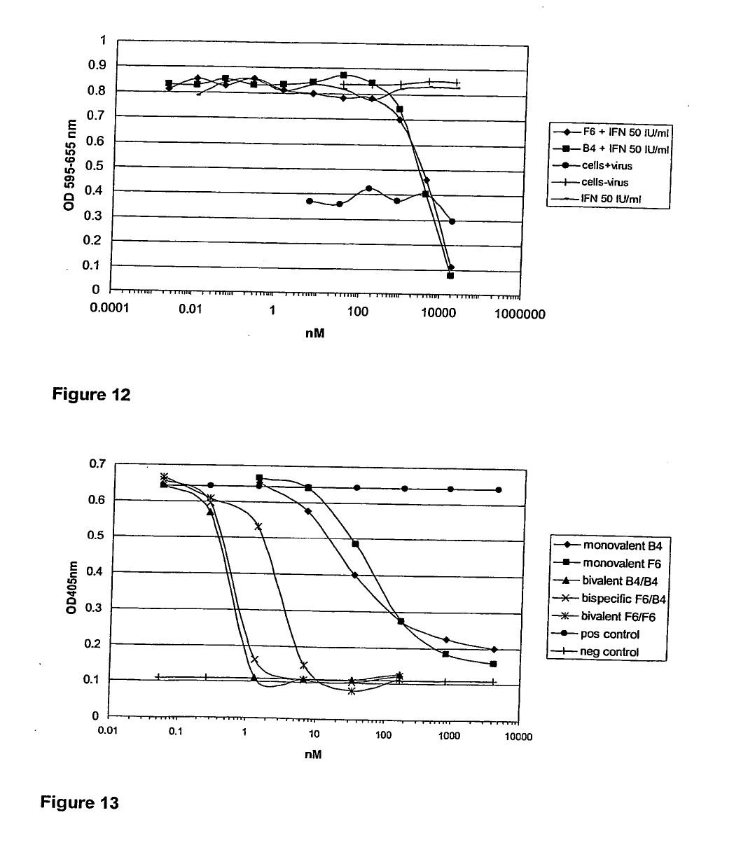

On day 1, FS4 cells were seeded at a concentration of 20,000 cells/well in a 96-well microtiter plate and grown in DMEM/10% FCS. On day 2, cells were treated with 50 or 5 IU/ml IFN-γ (expressed in CHO) pre-incubated for 1 hr at 37° C. with a dilution serie of VHH. On day 3, cells were infected with EMC virus (10³ particles/well). On day 4, 10 μl/well MTT (5 mg/ml) was added to detect viable cells. On day 5, 50 μl/well SDS (100 mg/ml) was added. Read-outs were done at 595-655 nm. Results for MP2F6SR and MP3B4SRA are presented in FIG. 12. Results for other isolated anti-human IFN-γ VHH are presented in Table 10.

### Example 12

### Construction of Bivalent and Bispecific VHH's

The DNA coding for MP3B4SRA and MP2F6SR VHH was amplified using a FR1 primer (5′-GAGGTBCARCTGCAGGASTCYGG-3′) and a FR4 primer (5′-GTGTGCGGCCGCTGAGGAGACRGTGACCWG-3′) introducing a Pst1 and a BstElI restriction site respectively. The PCR products were purified using a PCR purification kit (Qiagen). Half of the PCR product was digested with Pst1 at 37° C. for 1 hr and with BstElI at 60° C. for 1 hr, the other half with NotI for 1 hr at 37° C. and with SfiI for 1 hr at 50° C.

To construct a bivalent MP3B4SRA/MP3B4SRA, a bivalent MP-2F6SR/MP2F6SR and a bispecific MP3B4SRA/MP2F6SR, the PstI/BstElI digested products were purified over gel, ligated into pAX11 (PstI/BstElI) and transformed to WK6 Escherichia coli to obtain clones with a VHH at the C-terminus of the multicloning site. The clones were examined by PCR using the M13 reverse (5′-GGATAACAATTTCACACAGG-3′) and forward (5′-CACGACGTTGTAAAACGAC-3′) primers. From clones yielding a PCR fragment of 650 bp, DNA was prepared and digested with NotI for 1 hr at 37° C. and with SfiI for 1 hr at 50° C. Fragments were purified over gel and used as vector to clone the VHH (SfiI/NotI) at the N-terminus of the multicloning site. This yielded a bivalent MP3B4SRA/MP3B4SRA and a bispecific MP3B4SRA/MP2F6SR.

To clone the MP2F6SR VHH at the N-terminus another strategy was used as described above to get in frame expression of the C- and N-terminal VHH. MP2F6SR does not contain a hinge sequence. The hinge sequence was introduced by cloning the MP2F6SR VHH in pAX001 TNF 3E. pAX001 TNF 3E contains the coding sequence of a VHH in frame with a hinge sequence. This vector was digested with Pst1/BstElI to remove the irrelevant VHH, but not the hinge. The vector was gelpurified and used as acceptor vector to clone the DNA coding MP2F6SR. This procedure introduces MP2F6SR in frame with a hinge sequence. Subsequently this clone was digested with NotI for 1 hr at 37° C. and with SfiI for 1 hr at 50° C. The obtained fragments were cloned at the N-terminus of the multicloning site of the above described vector containing MP2F6SR at the C-terminus. This yielded a bivalent MP2F6SR/MP2F6SR. Constructs were examined by sequence analysis. Sequences are presented in Table 9.

### Example 13

### Functional Characterization of Bivalent and Bispecific VHH's: Inhibition of Binding of IFN-γ to the IFN-γ Receptor by a VHH in an In-House Receptor Binding Assay

Representative clones were expressed and purified as described in example 9 Purified VHH was analyzed for the ability to inhibit human IFN-γ/IFN-γ receptor interaction. Human IFN-γ receptor was coated at a concentration of 2 μg/ml overnight at 4° C. Plates were washed 5 times with PBS/0.05% Tween-20. Wells were blocked with 1% caseine overnight at 4° C. VHH was pre-incubated with 20 ng biotinylated human IFN-γ for 1 hr at room temperature. Mixture was applied to the wells and incubated for 2 hrs at room temperature. Plates were washed 5 times with PBS/0.05% Tween-20. Detection was performed using Extravidin-AP and pNPP as substrate. Plates were read at 405 nm after 30 minutes incubation at room temperature. Results are presented in FIG. 13.

### Example 14

### Functional Characterization of Bivalent and Bispecific VHH's: Inhibition of Binding of IFN-γ to the IFN-γ Receptor by a VHH in an in vitro Cell-Based Inhibition Assay

Purified bivalent and bispecific VHH were tested in cytotoxicity assays. Endotoxin was depleted from the samples using Tx-114. The samples were incubated for 30 minutes with 0.2% Tx-114. Subsequently, the mixture was incubated at 37° C. for 30 minutes and centrifuged for 10 minutes at 14,000 rpm. The upper phase was harvested and treated once more. There was no difference in binding in ELISA (example 7) or inhibition capacity (example 13) between Tx-114 treated and untreated VHH (data not shown).

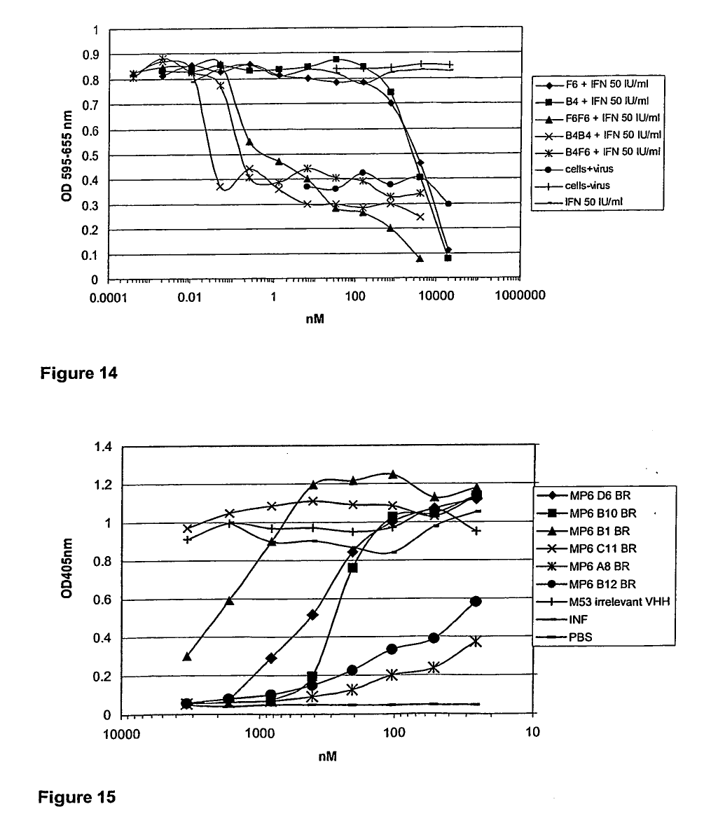

On day 1, FS4 cells were seeded at a concentration of 20,000 cells/well in a 96-well microtiter plate and grown in DMEM/10%FCS. On day 2, cells were treated with 50 or 5 IU/ml IFN-γ (expressed in CHO) pre-incubated for 1 hr at 37° C. with a dilution serie of VHH. On day 3, cells were infected with EMC virus (103 particles). On day 4, 10 μl/well MTT (5 mg/ml) was added to detect viable cells. On day 5, 50 μl/well SDS (100 mg/ml) was added. Read-outs were done at 595-655 nm. Results are presented in FIG. 14 and Table 11.

### Example 15

### Calculation of Homologies Between Anti-Target-Single Domain Antibodies of the Invention

The degree of amino acid sequence homology between anti-target single domain antibodies of the invention was calculated using the Bioedit Sequence Alignment Editor. The calculations indicate the proportion of identical residues between all of the sequences as they are aligned by ClustalW. (Thompson, J. D., Higgins, D. G. and Gibson, T. J. (1994) CLUSTAL W: improving the sensitivity of progressive multiple sequence alignment through sequence weighting, position specific gap penalties and weight matrix choice. Nucleic Acids Research, submitted, June 1994). Table 12 indicates the fraction homology between anti-serum albumin VHHs of the invention. Table 13 indicates the fraction homology between anti-TNF-alpha VHHs of the invention. Table 14 indicates the percentage homology between anti-IFN-gamma VHHs of the invention.

### Example 16

### Construction of a Bispecific Constructs Containing a VHH-CDR3 Fragment Fused to an Anti-Serum Albumin VHH

A functional portion, the CDR3 region of MP2F6SR, was amplified by using a sense primer located in the framework 4 region (F6 CRD3 Forward:CTGGCCCCAGAAGTCATACC) and an anti-sense primer located in the framework 3 region (F6 CDR3 Reverse primer:TGTGCATGTGCAGCAAACC). In order to fuse the CDR-3 fragment with the anti-serum albumin VHH MSA-21, a second round PCR amplification was performed with following primers:

F6 CDR3 Reverse primer Sfi1:
GTCCTCGCAACTGCGGCCCAGCCGGCCTGTGCATGTGCAGCAAACC
F6 CDR3 Forward primer Not1:
GTCCTCGCAACTGCGCGGCCGCCTGGCCCCAGAAGTCATACC

The PCR reactions were performed in 50 ml reaction volume using 50 pmol of each primer. The reaction conditions for the primary PCR were 11 min at 94° C., followed by 30/60/120 sec at 94/55/72° C. for 30 cycles, and 5 min at 72° C. All reaction were performed wit 2.5 mM MgCl2, 200 mM dNTP and 1.25U AmpliTaq God DNA Polymerase (Roche Diagnostics, Brussels, Belgium).

After cleavage of the VHH gene of MSA clones with restriction enzymes Pst1/BstElI the digested products were cloned in pAX11 to obtain clones with a VHH at the C-terminus of the multicloning site. The clones were examined by PCR using vector based primers. From clones yielding a 650 bp product, DNA was prepared and used as acceptor vector to clone the CDR3 of MP2F6SR, after cleavage of the PCR product with restriction enzymes Sfi1/Not1 to allow N-terminal expression of CDR3 in fusion with a MSA VHH.

These experiments show that the new class of VHH has bona fide binding and functional characteristics, thereby enabling their application for therapeutic purposes.

TABLE 1
Overview of the libraries, their diversity and % insert derived from
different llama's and tissues as described in Example 1 and 2

| Animal | Antigen | Source | Titer | | % Insert |
|---|---|---|---|---|---|
| Llama 5 | Human IFN-γ | PBL time 1 | 2.1 | 10⁸ | 94% |
| Llama 5 | Human IFN-γ | PBL time 2 | 7.5 | 10⁶ | 92% |
| Llama 5 | Human IFN-γ | Lymph node | 7.8 | 10⁸ | 100% |
| Llama 6 | Human IFN-γ | PBL time 1 | 1.15 | 10⁸ | 100% |
| Llama 6 | Human IFN-γ | PBL time 2 | 5.7 | 10⁷ | 96% |
| Llama 6 | Human IFN-γ | Lymph node | 2 | 10⁸ | 100% |
| Llama 22 | Human IFN-γ | PBL time 1 | 1.4 | 10⁸ | 79% |
| Llama 22 | Human IFN-γ | PBL time 2 | 2.4 | 10⁸ | 83% |
| Llama 22 | Human IFN-γ | Lymph node | 3 | 10⁷ | 92% |
| Llama 23 | Human IFN-γ | PBL time 1 | 3.7 | 10⁷ | 82% |
| Llama 23 | Human IFN-γ | PBL time 2 | 1 | 10⁸ | 71% |
| Llama 23 | Human IFN-γ | Lymph node | 1.3 | 10⁷ | 91% |
| Llama 29 | Mouse IFN-γ | PBL time 1 | 4.5 | 10⁷ | 96% |
| Llama 29 | Mouse IFN-γ | PBL time 2 | 1.6 | 10⁸ | 83% |
| Llama 29 | Mouse IFN-γ | Lymph node | 1.3 | 10⁸ | 100% |
| Llama 31 | Mouse IFN-γ | PBL time 1 | 1.6 | 10⁷ | 96% |
| Llama 31 | Mouse IFN-γ | PBL time 2 | 1 | 10⁸ | 83% |
| Llama 31 | Mouse IFN-γ | Lymph node | 8.6 | 10⁸ | 83% |

TABLE 2
Overview of screening experiments of different selections
for human IFN-γ specific VHH as described in Example 6-1

| | Experiment 1 | | Experiment 2 | | | Experiment 3 | |
|---|---|---|---|---|---|---|---|
| | (MP2 selection) | | (MP3 selection) | | | (MP4 selection) | |
| | Solid | | Solid | Solid | | Solid | |
| Selection | phase | Biotin. | phase | phase | Biotin. | phase | Biotin. |
| Elution | Receptor | Receptor | Receptor | Receptor | Receptor | Receptor | Receptor |
| | 1 hr | 1 hr | 1 hr | 1 hr + acid | 1 hr | ON | ON |
| Specific | 85% | 95% | 62% | 80% | 80% | 89% | 60% |
| binding | | | | | | | |
| Non-specific | 0% | 0% | 0% | 0% | 0% | 0% | 0% |
| binding | | | | | | | |
| % Insert | 90% | 100% | 90% | 95% | 100% | 100% | 91% |
| Diversity | 3 | 4 | 2 | 7 | 4 | 3 | 3 |
| (sequencing) | | | | | | | |

TABLE 3
Overview of screening experiments of selections for mouse
IFN-γ specific VHH as described in Example 6-2.

| Selection | Biotin. |
|---|---|
| Elution | Receptor 1 hr |
| Specific | 60% |
| binding | |
| Non-specific | 0% |
| binding | |
| % Insert | 98% |
| Diversity | 6 |
| (sequencing) | |

TABLE 4

Overview of amino acid sequence of human IFN-γ specific
VHH's as described in Example 8

| | | | |
|---|---|---|---|
| Seq. | | Seq. | |
| Family | Name | Id | Anti-human IFN gamma |

| | | | |
|---|---|---|---|
| 1 | MP3D2SRA | 1 | QVQLQDSGGGTVQAGGSLRLSCAASGRTFSDYAVGWFRQA |
| | | | PGKEREFVARILWTGASRSYANSVDGRFTVSTDNAKNTVY |
| | | | LQMNSLKPEDTAIYYCAALPSNIITTDYLRVYYWGQGTQV |
| | | | TVSS |
| 1 | MP3A3SR | 2 | QVQLQDSGGGTVQAGGSLRLSCAASGRTFSNYAVGWFRQA |
| | | | PGKEREFVARIKWSGGSRSYANSVDGRFTVSTDNAKNTVY |
| | | | LQMNSLKPEDTAIYYCA?LPSNIITTDYLRVYYWGQGTQV |
| | | | TVSS |
| 2 | MP3C5SR | 3 | QVQLQESGGGLVQAGGSLRLSCAAAGISGSVFSRTPMGWY |
| | | | RQAPGKQRELVAGILTSGATSYAESVKGRFTISRDNAKNT |
| | | | VYLQMNSLSPEDTAEYYCNTYPTWVLSWGQGTQVTVSS |
| 2 | MP3C1SR | 4 | QVQLQDSGGGLVQAGGSLRLSCAAAGISGSVFSRTPMGWY |
| | | | RQAPGKQRELVAGILSSGATVYAESVKGRFTISRDNAKNT |
| | | | VYLQMNSLSPEDTAEYYCNTYPTWVLSWGQGTQVTVSS |
| 2 | MP3G8SR | 5 | QVQLQESGGGLVQAGGSLRLSCAAAGISGSVFSRTPMGWY |
| | | | RQAPGKQRELVAGILSSGATAYAESVKGRFTISRDNAKNT |
| | | | VYLQMNSLSPEDTAEYYCNTYPTWVLSWGQGTQVTVSS |
| 3 | MP3D2BR | 6 | QVQLQESGGGLVQPGESLRLSCAASRGIFRFNAGGWYRQA |
| | | | PGKQRELVAFIGVDNTTRYIDSVKGRFTISRDNAKTTVYL |
| | | | QMNSLQPEDTAVYYCNKVPYIDWGQGTQVTVSS |
| 4 | MP3H6SRA | 7 | QVQLQESGGGLVQAGGSLRLSCAASGRTFSTYNMGWFRQA |
| | | | PGKEREFVAGISWNGGSIYYTSSVEGRFTISRDNAENTVY |
| | | | LQMNSLKPEDTGVYYCASKGRPYGVPSPRQGDYDYWGQGT |
| | | | QVTVSS |
| 4 | MP3B4SRA | 8 | QVQLQESGGGLVQAGGSLRLSCAASGRTFSTYNMGWFRQA |
| | | | PGKEREFVAGISWNGGSIYYTSSVEGRFTISRDNAENTVY |
| | | | LQMNSLKPEDTGVYYCASKGRPYGVPSPRQGDYDYWGQGT |
| | | | QVTVSS |
| 4 | MP4E4BR | 9 | QVQLQESGGGLVQAGGSLRLSCAASGRTFSIYNMGWFRQA |
| | | | PGKEREFVAAISWNGGSIYYTSSVEGRFTISRDNAINTVY |
| | | | LQMNSLKPEDTGVYYCASKGRPYGVPSPRQGEYDYWGQGT |
| | | | QVTVSS |
| 4 | MP4H8SR | 10 | QVQLQESGGGLVQAGGSLRLSCAASGRTFNIYNNGWFRQA |
| | | | PGKERDFVAAISWNGGSIYYTSSVEGRFTISRDNAENTVY |
| | | | LQMNSLKPEDTGVYYCASKGRPYGVPSPRQGDYDYWGQGT |
| | | | QVTVSS |
| 5 | MP2F6SR | 11 | QVKLEESGGGLVQAGGSLRLSCAASGRTFNNYNMGWFRQA |
| | | | PGKEREFVAAISWNGGSTYYDDSVKGRFTISRDNANNLVY |
| | | | LQMNSLNFEDTAVYYCACAANPYGIPQYRENRYDFWGQGT |
| | | | QVTVSS |
| 5 | MP3D1BR | 12 | QVQLQESGGGLVQAGGSLRLSCAASGRTFDNYNMGWFRQA |
| | | | PGKEREFVAAISWNGGSTYYDDSVKGRFTISRDNFQKLVY |
| | | | LQMNSLKLEDTAVYYCACAANPYGIPQYRENRYDFWGQGT |
| | | | QVTVSS |
| 6 | MP2B5BR | 13 | QVQLVESGGRLVQAGGSLRLSCIASGRTISDYAAGWFRQA |
| | | | PGKEREFASVTWGFGSTSYADSVKGRFTISPRDKAKDTVY |
| | | | LQMNTLEPDDTSVYYCASSPRYCAGYRCYVTASEFDSWGQ |
| | | | GTQVTVSS |
| 6 | MP2C1BR | 14 | QVKLEESGGRLVQAGGSLRLSCIASGRTISDYAAGWFRQA |
| | | | PGKEREFLASVSWGFGSTYYADSVKGRFTISRDTAKDTVY |
| | | | LQMNTLEPDDTSVYYCASSPRYCAGYRCYATASEFDSWGQ |
| | | | GTQVTVSS |
| 6 | MP4A12SR | 15 | QVQLQESGGRLVQAGGSLRLSCIASGRTISDYAAGWFRQA |
| | | | PGKEREFLASVTWGFGSTYYADSVKGRFTISRDKAKDTVY |
| | | | LQMNTLEPDDTSAYYCASSPRYCAGYRCYVTASEFDSWGP |
| | | | GTQVTVSS |
| 7 | MP3F4SRA | 16 | QVQLQDSGGGLVQAGDSLRLSCAASGRSFSSYGMGWFRQA |
| | | | PGKEHEFVAGIWRSGVSLYYTDSVKGRFTISRDDAKMTVS |
| | | | LQMNSLKPEDTAVYYCAAEATFPTWSRGRFADYDYRGQGT |
| | | | QVTVSS |
| 7 | MP3D3BR | 17 | QVQLQESGGGLVQAGDSLRLSCTASGRSFSSYGMGWFRQA |
| | | | PGKDHEFVAGIWRSGVSLYYADSVKGRFTISRDDAKMTVS |
| | | | LQMNGLKPEDTAVYYCAAEATFPTWNRGTFADYDYRGQGT |
| | | | QVTVSS |
| 7 | MP3E5BR | 18 | QVQLQESGGGLVQAGDSLRLSCAASGRSFSSYGMGWFRQA |
| | | | PGKEHEFVAGIWRSGVSLYYADSVKGRFTISRDDAKMTVS |
| | | | LQMNGLKPEDTAVYYCAAEATFPTWNRGSFADYDYRGQGT |
| | | | QVTVSS |
| 7 | MP3C7SRA | 19 | QVQLQESGGGLVQAGDSLRLSCAASGRSFSSYGMGWFRQA |
| | | | PGKEHEFVAGIWRSGVSLYYADSVKGRFTISRDDAKMTVS |
| | | | LQMNSLKPEDTAVYYCAAEATFPTWNRGRFADYDYSGQGT |
| | | | QVTVSS |
| 8 | MP2F1BR | 20 | AVQLVESGGGLVQTGDSLRLSCVASGGTFSRYAMGWFRQA |
| | | | PGKEREFVARIGYSGRSISYATSVEGRFAISRDNAKNTVY |
| | | | LQMNSLKPEDTAVYYCASLVSGTLYQADYWGQGTQVTVSS |
| 8 | MP2C5BR | 21 | QVQLVESGGGLVQTGDSLRLSCVASGGTFSRYAMGWFRQP |
| | | | PGKERDFVARIGYSGQSISYATSVEGRFAISRDNAKNTVY |
| | | | LQMNSLKPEDTAVYYCASLVSGTLYKPNYWGQGTQVTVSS |
| 9 | MP2C10BR | 22 | QVKLEESGGGLVQAGGSLRLSCAASGLTYTVGWFRQAPGK |
| | | | EREFVAAISWSGGSALYADSVKGRFTISRDNAKNTVYLQM |
| | | | GSLEPEDTAYYSCAAPGTRYYGSNQVNYNYWGQGTQVTVS |
| | | | S |
| 9 | MP2G5SR | 23 | QVKLEESGGGLVQAGDSLRLSCAASGLTYTVGWFRQAPGK |
| | | | EREFVAAIDWSGGSALYADSVKGRFTISRDMTKNTVYLQM |
| | | | GSLEPEDTAVYWCAAPGTRYHGRNQVNYNYWGQGTQVTVS |
| | | | S |
| 10 | MP3B1SRA | 24 | QVQLQESGGGLVQPGGSLRLSCAASGFTSSNYAMSWVRQA |
| | | | PGKGLEWVSSINSRTGSITYADSVKGRFTITLDNAKNTLY |
| | | | LQMNSLKPEDTAVYYCASRVDDRVSRGQGTQVTVSS |
| 11 | MP2F10SR | 25 | QVQLVESGGGLVQAGGSLRLSCAASGRTISSFRMGWFRRA |
| | | | PGEEREFVAFVRSNGTSTYYADSVEGRFTITRDNAKNTVY |
| | | | LRMDSLKPEDTAVYYCAAATRDYGGSFDYWGQGTQVTVSS |
| 11 | MP3A7SRA | 26 | QVQLQDSGGGLVQAGGSLRLSCAASGRTFSSFRMGWFRRA |
| | | | PGEEREFVAFVRSNGTSTYYADSVEGRFTITRDNAKNTVY |
| | | | LRMDSLKPEDTAVYYCAAATRDYGGSFDYWGQGTQVIVSS |
| 12 | MP4C10SR | 27 | QVQLQESGGGLVQPGGSLRLSCAASGFTVSNYAMSWVRQP |
| | | | PGKGIEWVSSINNRNDHITYADSVKGRFTIARDNANNILY |
| | | | LQMNSLKPEDTAVYYCASRVDDRVSRGQGTQVTVSS |
| 13 | MP4D5BR | 28 | QVQLQDSGGGLVQPGGSLRLSCAASGRTFSSYGMAWFRQA |
| | | | PGKERELVVAINRSGGATSYATSVRGRFTISRDNAKNTMY |
| | | | LQMNSLNPEDTAVYYCAARDPTRTYSSYFEYTYWGQGTQV |
| | | | TVSS |
| 14 | MP3F1SRA | 29 | QVQLQESGGGLVQAGGSLTLSCVASGRTISDYAVGWFRQA |
| | | | PGKEREFVASISWGGGFTAFADSMKGRFTISRDNAKNTVY |
| | | | LQTHTLEPDDTSVYYCASSRRYCTGYRCYATASEFDSWGQ |
| | | | GTQVTVSS |

TABLE 5

Overview of amino acid sequence of mouse IFN-gamma specific
VHH's as described in Example 8

| | | |
|---|---|---|
| | Seq | |
| Name | ID | Sequences |

| | | |
|---|---|---|
| MP6 D6 BR | 30 | QVQLQESGGGLVQAGGSLRLSCAVSGSIFSLLAMGWFRQA |
| | | PGKERELVASVSTHSNTNYADSVKGRFTISRDNAKNTVYL |
| | | QMNSLKPEDTAVYYCNAGGRYSARVYWGQGTQVTVSS |
| MP6 B1 BR | 31 | QVQLQESGGGLVQAGGSLRLSCAASGFTSDDYAIGWFRQA |
| | | PGKEREGVSCISSSDGVTYYADSVKGRFTISSDNAKNTVY |
| | | LQMNSLKPEDTAVYYCAADSLPLCFSGSYYHPYEYDYLGQ |
| | | GTQVTVSS |
| MP6 A8 BR | 32 | QVQLQESGGGLVQAGGSLRLSCAASGFTFDDYAIGWFRQA |
| | | PGKELEGVSMINSGGGSTYYADSVKGRFTISSDNAKNTVY |
| | | LQMNSLKPEDTAVYYCAADQNARLFRLWVVTGTGPVDNAL |
| | | DAWGQGTLVTVSS |
| MP6 B12 BR | 33 | QVQLQESGGGLVQAGGSLRLSCAASGFTFDDYDIGWFRQA |
| | | PGKEREEVSCISNIDGSTYYADSVKGRFTISSDNAKNTAY |
| | | LQMSSLKPEDTAVYYCAADIYVRCVHGLSPGYWGQGIQVT |
| | | VSS |
| MP6 C11 BR | 34 | QVQLQESGGGLVQAGGSLRLSCAASGRTFSTYAMGWFRQA |
| | | PGKEREFVAGITSSGGYTYYADSVKGRFTISRDNAKNTVY |
| | | LQMNSLKPEDTAVYYCAAGFRVGIALDLKGRYDYWGQGTQ |
| | | VTVSS |
| MP6 B10 BR | 35 | QVQLQDSGGGLVQLGGSLRLSCAISGRILGSYAVGWFRQA |
| | | PGKERQFVAAIGWSYGNTYYADSVKGRFTISRDNAKNTVY |
| | | LQINSLKPEDTAVYYCAAGDTYLTGRPNEYAYWGQGTQVT |
| | | VSS |

TABLE 6
Overview of IC50 of different IFN-γ specifc
VHH as described in Example 10

| | Mas- | | | | | |
|---|---|---|---|---|---|---|
| Se- | ter | | | | | |
| quence | plate | | Se- | | | IC50 |
| family | (MP) | Clone | lection | Elution | Name | (μg/ml) |
| 1 | 3 | A3 | Solid | Receptor | MP3A3SR | 0.065 |
| 2 | 3 | C1 | Solid | Receptor | MP3C1SR | >200 |
| 3 | 3 | D2 | Biotine | Receptor | MP3D2BR | 4 |
| 4 | 3 | B4 | Solid | Receptor + | MP3B4SRA | 0.35 |
| | | | | acid | | |
| 5 | 2 | F6 | Solid | Receptor | MP2F6SR | 1.75 |
| 6 | 2 | B5 | Biotine | Receptor | MP2B5BR | 8 |
| 7 | 3 | C7 | Solid | Receptor + | MP3C7SRA | >200 |
| | | | | acid | | |
| 8 | 2 | F1 | Biotine | Receptor | MP2F1BR | 8 |
| 9 | 2 | C10 | Biotine | Receptor | MP2C10BR | 3 |
| 10 | 3 | B1 | Solid | Receptor + | MP3B1SRA | 20 |
| | | | | acid | | |
| 11 | 3 | A7 | Solid | Receptor + | MP3A7SRA | 5 |
| | | | | acid | | |
| 12 | 4 | C10 | Solid | Receptor | MP4C10SR | >200 |
| | | | | ON | | |
| 13 | 4 | D5 | Biotine | Receptor | MP4D5BR | >200 |
| | | | | ON | | |
| 14 | 3 | F1 | Solid | Receptor + | MP3F1SRA | 8 |
| | | | | acid | | |

TABLE 7
Overview of Anti-mouse serum albumin/anti-human IFN-gamma binders

| | | |
|---|---|---|
| NAME | SEQ ID | SEQUENCE |

| | | |
|---|---|---|
| | | Anti-mouse serum albumin |
| MSA21 | 36 | QVQLQESGGGLVQPGGSLRLSCEASGFTFSRFGMTWVRQAPGKGVEWVSG |
| | | ISSLGDSTLYADSVKGRFTISRDNAKNTLYLQMNSLKPEDTAVYYCTIGG |
| | | SLNPGGQGTQVTVSS |
| MSA24 | 37 | QVQLQESGGGLVQPGNSLRLSCAASGFTFRNFGMSWVRQAPGKEPEWVSS |
| | | ISGSGSNTIYADSVKDRFTISRDNAKSTLYLQMNSLKPEDTAVYYCTIGG |
| | | SLSRSSQGTQVTVSS |
| MSA210 | 38 | QVQLQESGGGLVQPGGSLPLTCTASGFTFSSFGMSWVRQAPGKGLEWVSA |
| | | ISSDSGTKNYADSVKGRFTISRDNAKKMLFLQMNSLRPEDTAVYYCVIGR |
| | | GSPSSQGTQVTVSS |
| MSA212 | 39 | QVQLQESGGGLVQPGGSLPLTCTASGFTFRSFGMSWVRQAPGKGLEWVSA |
| | | ISADGSDKRYADSVKGRFTISRDNGKKMLTLDMNSLKPEDTAVYYCVIGR |
| | | GSPASQGTQVTVSS |
| MSAc16 | 62 | AVQLVESGGGLVQAGDSLRLSCVVSGTTFSSAAMGWFRQAPGKEREFVGA |
| | | IKWSGTSTYYTDSVKGRFTISRDNVKNTVYLQMNNLKPEDTGVYTCAADR |
| | | DRYRDRMGPMTTTDFRFWGQGTGVTVSS |
| MSAc112 | 63 | QVKLEESGGGLVQTGGSLRLSCAASGRTFSSFAMGWFRQAPGREREFVAS |
| | | IGSSGITTNYADSVKGRFTISRDNAKNTVYLQMNSLKPEDTGLCYCAVNR |
| | | YGIPYRSGTQYQNWGQGTQVTVSS |
| MSAc110 | 64 | EVQLEESGGGLVQPGGSLRLSCAASGLTFNDYAMGWYRQAPGKERDMVAT |
| | | ISIGGRTYYADSVKGRFTISRDNAKNTVYLQMNSLKPEDTAIYYCVAHRQ |
| | | TVVRGPYLLWGQGTQVTVSS |
| MSAc114 | 65 | QVQLVESGGKLVQAGGSLRLSCAASGRTFSNYAMGWFRQAPGKEREFVAG |
| | | SGRSNSYNYYSDSVKGRFTISRDNAKNTVYLQMNSLKPEDTAVYYCAAST |
| | | NLWPRDRNLYAYWGQGTQVTVSS |
| MSAc116 | 66 | EVQLVESGGGLVQAGDSLRLSCAASGRSLGIYRMGWFRQVPGKEREFVAA |
| | | ISWSGGTTRYLDSVKGRFTISRDSTKNAVYLQMNSLKPEDTAVYYCAVDS |
| | | SGRLYWTLSTSYDYWGQGTQVTVSS |
| MSAc119 | 67 | QVQLVEFGGGLVQAGDSLRLSCAASGRSLGIYKMAWFRQVPGKEREFVAA |
| | | ISWSGGTTRYIDSVKGRFTLSRDNTKNMVYLQMNSLKPDDTAVYYCAVDS |
| | | SGRLYWTLSTSYDYWGQGTQVTVSS |
| MSAc15 | 68 | EVQLVESGGGLVQAGGSLSLSCAASGRTFSPYTMGWFRQAPGKEREFLAG |
| | | VTWSGSSTFYGDSVKGRFTASRDSAKNTVTLEMNSLNPEDTAVYYCAAAY |
| | | GGGLYRDPRSYDYWGRGTQVTVSS |
| MSc111 | 69 | AVQLVESGGGLVQAGGSLRLSCAASGFTLDAWPIAWFRQAPGKEREGVSC |
| | | IRDGTTYYADSVKGRFTISSDNANNTVYLQTNSLKPEDTAVYYCAAPSGP |
| | | ATGSSHTFGIYWNLRDDYDNWGQGTQVTVSS |
| MSAc115 | 70 | EVQLVESGGGLVQAGGSLRLSCAASGFTFDHYTIGWFRQVPGKEREGVSC |
| | | ISSSDGSTYYADSVKGRFTISSDNAKNTVYLQMNTLEPDDTAVYYCAAGG |
| | | LLLRVEELQASDYDYWGQGIQVTVSS |
| MSAc18 | 71 | AVQLVDSGGGLVQPGGSLRLSCTASGFTLDYYAIGWFRQAPGKEREGVAC |
| | | ISNSDGSTYYGDSVKGRFTISRDNAKTTVYLQMNSLKPEDTAVYYCATAD |
| | | RHYSASHHPFADFAFNSWGQGTQVTVSS |
| MSAc17 | 72 | EVQLVESGGGLVQAGGSLRLSCAAYGLTFWRAAMAWFRRAPGKERELVVA |
| | | RNWGDGSTRYADSVKGRFTISRDNAKNTVYLQMNSLKPEDTAVYYCAAVR |
| | | TYGSATYDIWGQGTQVTVSS |
| MSAc120 | 73 | EVQLVESGGGLVQDGGSLRLSCIFSGRTFANYAMGWFRQAPGKEREFVAA |
| | | INRNGGTTNYADALKGRFTISRDNTKNTAFLQMNSLKPDDTAVYYCAARE |
| | | WPFSTIPSGWRYWGQGTQVTVSS |
| MSAc14 | 74 | DVQLVESGGGWVQPGGSLRLSCAASGPTASSHAIGWFRQAPGKEREFVVG |
| | | INRGGVTRDYADSVKGRFAVSRDNVKNTVYLQMNRLKPEDSAIYICAARP |
| | | EYSFTAMSKGDMDYWGKGTLVTVSS |
| | | Anti-mouse serum albumin/anti -IFN-ganmia |
| MSA | 40 | QVQLQESGGGLVQPGGSLRLSCEASGFTFSRFGMTWVRQAPGKGVEW |
| 21/ | | VSGISSLGDSTLYADSVKGRFTISRDNAKNTLYLQMNSLKPEDTAVY |
| MP2F6S | | YCTIGGSLNPGGQGTQVTVSSEPKTPKPQPAAAQVKLEESGGGLVQA |
| R | | GGSLRLSCAASGRTFNNYNMGWFRQAPGKEREFVAAISWNGGSTYYD |
| | | DSVKGRFTISRDNANNLVYLQMNSLNFEDTAVYYCACAANPYGIPQY |
| | | RENRYDFWGQGTQVTVSS |
| MSA | 41 | QVQLQESGGGLVQPGNSLRLSCAASGFTFRNFGMSWVRQAPGKEPEW |
| 24/ | | VSSISGSGSNTIYADSVKDRFTISRDNAKSTLYLQMNSLKPEDTAVY |
| MP2F1B | | YCTIGGSLSRSSQGTQVTVSSEPKTPKPQPAAAAVQLVESGGGLVQT |
| R | | GDSLRLSCVASGGTFSRYAMGWFRQAPGKEREFVARIGYSGRSISYA |
| | | TSVEGRFAISRDNAKNTVYLQMNSLKPEDTAVYYCASLVSGTLYQAD |
| | | YWGQGTQVTVSS |
| MSA | 42 | QVQLQESGGGLVQPGGSLRLTCTASGFTFSSFGMSWVRQAPGKGLEW |
| 210/ | | VSAISSDSGTKNYADSVKGRFTISRDNAKKMLFLQMNSLRPEDTAVY |
| MP3B4S | | YCVIGRGSPSSQGTQVTVSSEPKTPKPQPAAAQVQLQESGGGLVQAGGSL |
| RA | | RLSCAASGRTFSTYNMGWFRQAPGKEREFVAGISWNGGSIYYTSSVEGRF |
| | | TISRDNAENTVYLQMNSLKPEDTGVYYCASKGRPYGVPSPRQGDYDYWGQ |
| | | GTQVTVSS |

TABLE 8

Amino acid sequence listing of the peptides of aspects of
present invention directed against TNF-alpha.

| | | |
|---|---|---|
| | SEQ | |
| | ID | |
| NAME | NO | SEQUENCE |

| | | |
|---|---|---|
| VHH#1A | 43 | QVQLQESGGGLVQPGGSLRLSCATSGFDFSVSWMYWVRQAPGKGLEWVSEI |
| | | NTNGLITKYVDSVKGRFTISRDNAKNTLYLQMDSLIPEDTALYYCARSPSG |
| | | SFRGQGTQVTVSS |
| VHH#7B | 44 | QVQLQESGGGLVQPGGSLRLSCAASGSIFRVNAMGWYRQVPGNQREFVAII |
| | | TSGDNLNYADAVKGRFTISTDNVKKTVYLQMNVLKPEDTAVYYCNAILQTS |
| | | RWSIPSNYWGQGTQVTVSS |
| VHH#2B | 45 | QVQLQESCGGLVQPGGSLPWSCATSGFTFSDYWMYWVRQAPGKGLEWVSTV |
| | | NTNGLITRYADSVKGRFTISRDNAKYTLYLQMNSLKSEDTAVYYCTKVVPP |
| | | YSDDSRTNADWGQGTQVTVSS |
| VHH#3E | 46 | QVQLQESGGGLVQPGGSLRLSCAASGRTFSDHSGYTYTIGWFRQAPGKERE |
| | | FVARIYWSSGNTYYADSVKGRFAISRDIAKNTVDLTMNNLEPEDTAVYYCA |
| | | ARDGIPTSRSVESYNYWGGTQVTVSS |
| VHH#3G | 47 | QVQLQDSGGGLVQAGGSLRLSCAVSGRTFSAHSVYTMGWFRQAPGKEREFV |
| | | ARIYWSSANTYYADSVKGRFTISRDNAKNTVDLLMNSLKPEDTAVYYCAAR |
| | | DGIPTSRTVGSYNYWGQGTQVTVSS |
| VHH#10A | 48 | QVQLQESGGGLVQPGGSLRLSCAASGSIFRVNAMGWYRQVPGNQREFVAII |
| | | TSSDTNDTTNYADAVKGRFTISTDNVKKTVYLQMNVLKPEDTAVYYCNAVL |
| | | QTSRWSIPSNYWGQGTQVTVSS |
| VHH#2G | 49 | QVQLQDSGGGLVQAGGSLRLSCTTSGRTISVYAMGWFRQAPGKEREFVASI |
| | | SGSGAITPYADSVKGRFTISRDNAKNTVYLQMNSLNPEDTAVYYCAASRYA |
| | | RYRDVHAYDYWGQGTQVTVSS |
| VHH#1F | 50 | QVQLQDSGGGLVQAGGSLRLSCAASTRTFSRYVVGWFRQAPGKEREFVATI |
| | | SWNGEHTYYADSVKGRYTISRDNAKNTVYLQMGSLKPEDTAVYYCAARSFW |
| | | GYNVEQRDFGSWGQGTPVTVSS |
| VHH#9C | 51 | QVQLQESGGGLVQPGGSLRLSCAASGSIFRVNAMGWYRQVPGNQREFVAII |
| | | TNDTTNYADAVKGRFTISTDNVKKTVYLQMNVLKPEDTAVYYCNTVLQTSR |
| | | WNIPTNYWSQGTQVTVSS |
| VHH#11E | 52 | QVQLQESGGGLVQPGGSLRLSCAASGSIFRVNAMGWYRQVPGNQREFVAII |
| | | SGDTTNYADAVKGRFTISTDNVKKTVYLQMNVLESEDTAVYYCNAVLQTSR |
| | | WSIPSNYWGQGTQVTVSS |
| VHH#10C | 53 | QVQLQDSGGGLVQPGGSLRLACVASGSIFSIDVMGWYRQAPGQQRELVATI |
| | | TNSWTTNYADSVKGRFTISRDNAKNVVYLQMNSLKLEDTAVYYCNARRWYQ |
| | | PEAWGQGTQVTVSS |
| VHH#4B | 54 | QVQLQDSGGGLVQPGGSLRLSCAASGFTFSTHWMYWVRQAPGKGLEWVSTI |
| | | NTNGLITDYIHSVKGRFTISRDNAKNTLYLQMNSLKSEDTAVYYCALNQAG |
| | | LSRGQGTQVTVSS |
| VHH#10D | 55 | QVQLQESGGGLVQAGGSLRLSCAASRRTFSGYAMGWFRQAPGKEREFVAVV |
| | | SGTGTYAYYADSVKGRFTISRDNAENTVYLQMNSLKPEDTGLYYCAVGPSS |
| | | SRWYYRGASLVDYWGKGTLVTVSS |
| VHH#12B | 56 | QVQLQESGGGLVQPGGSLRLSCAASGFEFENHWMYWVRQAPGKGLEWVSTV |
| | | NTNGLITRYADSVKGRFTISRDNAKYTLYLQMNSLKSEDTAVYYCTKVLPP |
| | | YSDDSRTNADWGQGTQVTVSS |
| VHH 9E | 57 | EVQLVESGGGLVQAGGSLRLSCAASGGTLSSYITGWFRQAPGKEREFVGAV |
| | | SWSSSTIVYADSVEGRFTISRDNNQNTVYLQMDSLKPEDTAVYYCAARPYQ |
| | | KYNWASASYNVWGQGTQVTVSS |
| VHH 3F | 58 | QVQLQDSGGGLVQAGGSLRLSCAASGGTFSSIIMAWFRQAPGKEREFVGAV |
| | | SWSGGTTVYADSVLGRFEISRDSARKSVYLQMNSLKPEDTAVYYCAARPYQ |
| | | KYNWASASYNVWGQGTQVTVSS |

TABLE 9
Overview of amino acid sequence of bivalent and bispecific human
IFN-γ specific VHH's as described in Example 12

| | | |
|---|---|---|
| | SEQ | |
| | ID | |
| NAME | NO | SEQUENCE |

| | | |
|---|---|---|
| MP2F6S | 59 | QVKLEESGGGLVQAGGSLRLSCAASGRTFNNYNMGWFRQAPGKEREFVAAI |
| R/MP2F6 | | SWNGGSTYYDDSVKGRFTISRDNANNLVYLQMNSLNFEDTAVYYCACAANP |
| SR | | YGIPQYRENRYDFWGQGTQVTVSSEPKTPKPQPAAAQVKLEESGGGLVQAG |
| | | GSLRLSCAASGRTFNNYNMGWFRQAPGKEREFVAAISWNGGSTYYDDSVKG |
| | | RFTISRDNANNLVYLQMNSLNFEDTAVYYCACAANPYGIPQYRENRYDFWG |
| | | QGTQVTVSS |
| MP3B4S | 60 | QVQLQESGGGLVQAGGSLRLSCAASGRTFSTYNMGWFRQAPGKEREFVAGI |
| RA/MP3 | | SWNGGSIYYTSSVEGRFTISRDNAENTVYLQMNSLKPEDTGVYYCASKGRP |
| B4SRA | | YGVPSPRQGDYDYWGQGTQVTVSSEPKTPKPQPAAAQVQLQESGGGLVQAG |
| | | GSLRLSCAASGRTFSTYNMGWFRQAPGKEREFVAGTSWNGGSIYYTSSVEG |
| | | RFTISRDNAENTVYLQMNSLKPEDTGVYYCASKGRPYGVPSPRQGDYDYWG |
| | | QGTQVTVSS |
| MP3B4S | 61 | QVQLQESGGGLVQAGGSLRLSCAASGRTFSTYNMGWFRQAPGKEREFVAGI |
| RA/MP2 | | SWNGGSIYYTSSVEGRFTISRDNAENTVYLQMNSLKPEDTGVYYCASKGRP |
| F6SR | | YGVPSPRQGDYDYWGQGTQVTVSSEPKTPKPQPAAAQVKLEESGGGLVQAG |
| | | GSLRLSCAASGRTFNNYNMGWFRQAPGKEREFVAAISWNGGSTYYDDSVKG |
| | | RFTISRDNANNLVYLQMNSLNFEDTAVYYCACAANPYGIPQYRENRYDFWG |
| | | QGTQVTVSS |

TABLE 10
Overview of IC50 of different monovalent human IFN-γ
specific VHH as described in example 11.

| | Mas- | | | | | |
|---|---|---|---|---|---|---|
| Se- | ter- | | | | | |
| quence | plate | | Se- | | | IC50 |
| family | (MP) | Clone | lection | Elution | Name | (nM) |
| 1 | 3 | A3 | Solid | Receptor | MP3A3SR | 17,000 |
| 2 | 3 | C1 | Solid | Receptor | MP3C1SR | 9,000 |
| 3 | 3 | D2 | Biotine | Receptor | MP3D2BR | 9,000 |
| 4 | 3 | B4 | Solid | Receptor + | MP3B4SRA | 2,000 |
| | | | | acid | | |
| 5 | 2 | F6 | Solid | Receptor | MP2F6SR | 1,500 |
| 6 | 2 | B5 | Biotine | Receptor | MP2B5BR | 2,000 |
| 7 | 3 | C7 | Solid | Receptor + | MP3C7SRA | 7,500 |
| | | | | acid | | |
| 8 | 2 | F1 | Biotine | Receptor | MP2F1BR | 5,000 |
| 9 | 2 | C10 | Biotine | Receptor | MP2C10BR | 25,000 |
| 10 | 3 | B1 | Solid | Receptor + | MP3B1SRA | 9,000 |
| | | | | acid | | |
| 11 | 3 | A7 | Solid | Receptor + | MP3A7SRA | 9,000 |
| | | | | acid | | |
| 12 | 4 | C10 | Solid | Receptor | MP4C10SR | 200,000 |
| | | | | ON | | |
| 13 | 4 | D5 | Biotine | Receptor | MP4D5BR | 15,000 |
| | | | | ON | | |
| 14 | 3 | F1 | Solid | Receptor + | MP3F1SRA | 9,000 |
| | | | | acid | | |

TABLE 11
Overview of IC50 of bivalent/bispecific human IFN-γ specific
VHH and IgG/Fab derived from neutralizing polyclonal goat
anti-human IFN-γ serum as described in example 14

| | | IC50 |
|---|---|---|
| Format | Name | (nM) |
| Bivalent | MP2F6SR/MP2F6SR | 0.150 |
| Bivalent | MP3B4SRA/MP3B4SRA | 0.030 |
| Bispecific | MP3B4SRA/MP2F6SR | 0.120 |
| IgG | Goat anti-human IFN-γ polyclonal (Advanced | 70 |
| | Biotherapy Inc) | |
| Fab | Goat anti-human IFN-γ polyclonal (Advanced | 70 |
| | Biotherapy Inc) | |

TABLE 12
Fractional homologies between the amino acid sequences
of anti-mouse serum albumin VHHs of the invention.

| SEQ | MSA21 | MSA24 | MSA210 | MSA212 |
|---|---|---|---|---|
| MSA21 | 1.000 | 0.834 | 0.800 | 0.782 |
| MSA24 | — | 1.000 | 0.782 | 0.791 |
| MSA210 | — | — | 1.000 | 0.903 |
| MSA212 | — | — | — | 1.000 |

TABLE 13
Fractional homologies between anti-TNF-alpha VHHs of the invention.

| SEQ | VHH#1A | VHH#7B | VHH#2B | VHH#3E | VHH#3G | VHH#10A | VHH#2G | VHH#1F |
|---|---|---|---|---|---|---|---|---|
| VHH#1A | 1.000 | 0.601 | 0.764 | 0.596 | 0.622 | 0.600 | 0.682 | 0.629 |
| VHH#7B | — | 1.000 | 0.604 | 0.635 | 0.645 | 0.943 | 0.653 | 0.616 |
| VHH#2B | — | — | 1.000 | 0.620 | 0.645 | 0.611 | 0.682 | 0.661 |
| VHH#3E | — | — | — | 1.000 | 0.875 | 0.641 | 0.713 | 0.689 |
| VHH#3G | — | — | — | — | 1.000 | 0.651 | 0.779 | 0.740 |
| VHH#10A | — | — | — | — | — | 1.000 | 0.658 | 0.614 |
| VHH#2G | — | — | — | — | — | — | 1.000 | 0.741 |
| VHH#1F | — | — | — | — | — | — | — | 1.000 |
| VHH#9C | — | — | — | — | — | — | — | — |
| VHH#11E | — | — | — | — | — | — | — | — |
| VHH#10C | — | — | — | — | — | — | — | — |
| VHH#4B | — | — | — | — | — | — | — | — |
| VHH#10D | — | — | — | — | — | — | — | — |
| VHH#12B | — | — | — | — | — | — | — | — |
| VHH#9E | — | — | — | — | — | — | — | — |
| VHH#3F | | | | | | | | |
| SEQ | VHH#9C | VHH#11E | VHH#10C | VHH#4B | VHH#10D | VHH#12B | VHH#9E | VHH#3F |
| VHH#1A | 0.609 | 0.601 | 0.614 | 0.818 | 0.642 | 0.747 | 0.596 | 0.604 |
| VHH#7B | 0.933 | 0.933 | 0.719 | 0.593 | 0.614 | 0.620 | 0.616 | 0.624 |
| VHH#2B | 0.629 | 0.620 | 0.637 | 0.796 | 0.634 | 0.951 | 0.620 | 0.645 |
| VHH#3E | 0.620 | 0.643 | 0.612 | 0.604 | 0.648 | 0.596 | 0.674 | 0.682 |
| VHH#3G | 0.637 | 0.637 | 0.653 | 0.645 | 0.689 | 0.622 | 0.708 | 0.716 |
| VHH#10A | 0.935 | 0.935 | 0.725 | 0.592 | 0.612 | 0.626 | 0.622 | 0.637 |
| VHH#2G | 0.653 | 0.669 | 0.685 | 0.666 | 0.746 | 0.650 | 0.701 | 0.717 |
| VHH#1F | 0.616 | 0.616 | 0.664 | 0.661 | 0.714 | 0.645 | 0.709 | 0.717 |
| VHH#9C | 1.000 | 0.941 | 0.743 | 0.601 | 0.622 | 0.645 | 0.600 | 0.616 |
| VHH#11E | — | 1.000 | 0.719 | 0.601 | 0.622 | 0.637 | 0.608 | 0.624 |
| VHH#10C | — | — | 1.000 | 0.650 | 0.606 | 0.637 | 0.600 | 0.632 |
| VHH#4B | — | — | — | 1.000 | 0.611 | 0.796 | 0.588 | 0.629 |
| VHH#10D | — | — | — | — | 1.000 | 0.619 | 0.674 | 0.674 |
| VHH#12B | — | — | — | — | — | 1.000 | 0.604 | 0.637 |
| VHH#9E | — | — | — | — | — | — | 1.000 | 0.854 |
| VHH#3F | | | | | | | | 1.000 |

TABLE 14
Percentage homologies between anti-IFN-gamma VHHs of the invention.

| | % Homology | | | | | | | | |
|---|---|---|---|---|---|---|---|---|---|
| | MP3D2SRA | MP3A3SR | MP3C5SR | MP3C1SR | MP3G8SR | P3D2BR | MP3H6SRA | MP3B4SRA | MP4E4BR |
| MP3D2SRA | X | 96 | 66 | 66 | 66 | 62 | 71 | 71 | 71 |
| MP3A3SR | — | X | 66 | 66 | 66 | 62 | 72 | 72 | 72 |
| MP3C5SR | — | — | X | 97 | 98 | 73 | 65 | 65 | 64 |
| MP3C1SR | — | — | — | X | 98 | 72 | 64 | 64 | 64 |
| MP3G8SR | — | — | — | — | X | 73 | 65 | 65 | 64 |
| MP3D2BR | — | — | — | — | — | X | 63 | 63 | 63 |
| MP3H6SRA | — | — | — | — | — | — | X | 100 | 97 |
| MP3B4SRA | — | — | — | — | — | — | — | X | 97 |
| MP4E4BR | — | — | — | — | — | — | — | — | X |
| MP4H8SR | — | — | — | — | — | — | — | — | — |
| MP2F6SR | — | — | — | — | — | — | — | — | — |
| MP3D1BR | — | — | — | — | — | — | — | — | — |
| MP2B5BR | — | — | — | — | — | — | — | — | — |
| MP2C1BR | — | — | — | — | — | — | — | — | — |
| MP4A12SR | — | — | — | — | — | — | — | — | — |
| MP3F4SRA | — | — | — | — | — | — | — | — | — |
| MP3D3BR | — | — | — | — | — | — | — | — | — |
| MP3E5BR | — | — | — | — | — | — | — | — | — |
| MP3C7SRA | — | — | — | — | — | — | — | — | — |
| MP2F1BR | — | — | — | — | — | — | — | — | — |
| MP2C5BR | — | — | — | — | — | — | — | — | — |
| MP2C10BR | — | — | — | — | — | — | — | — | — |
| MP2G5SR | — | — | — | — | — | — | — | — | — |
| MP3B1SRA | — | — | — | — | — | — | — | — | — |
| MP2F10SR | — | — | — | — | — | — | — | — | — |
| MP3A7SRA | — | — | — | — | — | — | — | — | — |
| MP4C10SR | — | — | — | — | — | — | — | — | — |
| MP4D5BR | — | — | — | — | — | — | — | — | — |
| MP3F1SRA | — | — | — | — | — | — | — | — | — |
| MP6D6BR | — | — | — | — | — | — | — | — | — |
| MP6B1BR | — | — | — | — | — | — | — | — | — |
| MP6A8BR | — | — | — | — | — | — | — | — | — |
| MP6B12BR | — | — | — | — | — | — | — | — | — |
| MP6C11BR | | | | | | | | | |
| MP6B10BR | | | | | | | | | |

| | % Homology | | | | | | | | |
|---|---|---|---|---|---|---|---|---|---|
| | MP4H8SR | MP2F6SR | MP3D1BR | MP2B5BR | MP2C1BR | MP4A12SR | MP3F4SRA | MP3D3BR | MP3E5BR |
| MP3D2SRA | 70 | 68 | 69 | 65 | 63 | 64 | 68 | 66 | 67 |
| MP3A3SR | 71 | 70 | 71 | 65 | 63 | 64 | 68 | 66 | 67 |
| MP3C5SR | 63 | 63 | 63 | 60 | 58 | 59 | 64 | 64 | 65 |
| MP3C1SR | 62 | 62 | 62 | 58 | 57 | 58 | 65 | 64 | 64 |
| MP3G8SR | 63 | 63 | 63 | 59 | 58 | 59 | 64 | 64 | 65 |
| MP3D2BR | 62 | 63 | 64 | 59 | 58 | 58 | 62 | 61 | 62 |
| MP3H6SRA | 97 | 80 | 81 | 67 | 68 | 67 | 75 | 71 | 73 |
| MP3B4SRA | 97 | 80 | 81 | 67 | 68 | 67 | 75 | 71 | 73 |
| MP4E4BR | 97 | 81 | 82 | 68 | 69 | 68 | 73 | 70 | 71 |
| MP4H8SR | X | 81 | 81 | 66 | 66 | 66 | 72 | 69 | 71 |
| MP2F6SR | — | X | 94 | 65 | 68 | 64 | 70 | 67 | 69 |
| MP3D1BR | — | — | X | 65 | 66 | 65 | 71 | 69 | 71 |
| MP2B5BR | — | — | — | X | 95 | 97 | 63 | 64 | 64 |
| MP2C1BR | — | — | — | — | X | 95 | 63 | 64 | 64 |
| MP4A12SR | — | — | — | — | — | X | 63 | 64 | 64 |
| MP3F4SRA | — | — | — | — | — | — | X | 94 | 96 |
| MP3D3BR | — | — | — | — | — | — | — | X | 98 |
| MP3E5BR | — | — | — | — | — | — | — | — | X |
| MP3C7SRA | — | — | — | — | — | — | — | — | — |
| MP2F1BR | — | — | — | — | — | — | — | — | — |
| MP2C5BR | — | — | — | — | — | — | — | — | — |
| MP2C10BR | — | — | — | — | — | — | — | — | — |
| MP2G5SR | — | — | — | — | — | — | — | — | — |
| MP3B1SRA | — | — | — | — | — | — | — | — | — |
| MP2F10SR | — | — | — | — | — | — | — | — | — |
| MP3A7SRA | — | — | — | — | — | — | — | — | — |
| MP4C10SR | — | — | — | — | — | — | — | — | — |
| MP4D5BR | — | — | — | — | — | — | — | — | — |
| MP3F1SRA | — | — | — | — | — | — | — | — | — |
| MP6D6BR | — | — | — | — | — | — | — | — | — |
| MP6B1BR | — | — | — | — | — | — | — | — | — |
| MP6A8BR | — | — | — | — | — | — | — | — | — |
| MP6B12BR | — | — | — | — | — | — | — | — | — |
| MP6C11BR | | | | | | | | | |
| MP6B10BR | | | | | | | | | |

| | % Homology | | | | | | | | |
|---|---|---|---|---|---|---|---|---|---|
| | MP3C7SRA | MP2F1BR | MP2C5BR | MP2C10BR | MP2G5SR | MP3B1SRA | MP2F10SR | MP3A7SRA | MP4C10SR |
| MP3D2SRA | 68 | 71 | 70 | 68 | 67 | 63 | 67 | 68 | 60 |
| MP3A3SR | 68 | 72 | 72 | 69 | 67 | 64 | 66 | 67 | 60 |
| MP3C5SR | 66 | 65 | 65 | 65 | 63 | 63 | 64 | 64 | 61 |
| MP3C1SR | 65 | 64 | 63 | 64 | 62 | 63 | 64 | 65 | 60 |
| MP3G8SR | 66 | 65 | 64 | 65 | 63 | 63 | 65 | 65 | 61 |
| MP3D2BR | 63 | 64 | 63 | 63 | 63 | 64 | 63 | 63 | 63 |
| MP3H6SRA | 75 | 73 | 71 | 73 | 71 | 66 | 75 | 75 | 63 |
| MP3B4SRA | 75 | 73 | 71 | 73 | 71 | 66 | 75 | 75 | 63 |
| MP4E4BR | 73 | 73 | 71 | 73 | 71 | 66 | 75 | 75 | 63 |
| MP4H8SR | 72 | 71 | 71 | 72 | 71 | 64 | 73 | 73 | 62 |
| MP2F6SR | 71 | 67 | 65 | 73 | 71 | 63 | 71 | 70 | 62 |
| MP3D1BR | 72 | 67 | 65 | 70 | 69 | 63 | 71 | 71 | 62 |
| MP2B5BR | 64 | 65 | 63 | 64 | 63 | 60 | 66 | 63 | 57 |
| MP2C1BR | 64 | 63 | 61 | 66 | 65 | 59 | 66 | 63 | 56 |
| MP4A12SR | 64 | 62 | 60 | 63 | 62 | 59 | 65 | 63 | 56 |
| MP3F4SRA | 97 | 69 | 67 | 68 | 68 | 62 | 67 | 69 | 60 |
| MP3D3BR | 96 | 70 | 68 | 67 | 67 | 62 | 67 | 67 | 60 |
| MP3E5BR | 98 | 70 | 68 | 68 | 69 | 63 | 68 | 68 | 60 |
| MP3C7SRA | X | 71 | 69 | 69 | 70 | 63 | 69 | 69 | 61 |
| MP2F1BR | — | X | 94 | 66 | 67 | 63 | 68 | 67 | 61 |
| MP2C5BR | — | — | X | 66 | 67 | 63 | 67 | 65 | 62 |
| MP2C10BR | — | — | — | X | 94 | 62 | 68 | 66 | 59 |
| MP2G5SR | — | — | — | — | X | 62 | 67 | 65 | 59 |
| MP3B1SRA | — | — | — | — | — | X | 66 | 65 | 91 |
| MP2F10SR | — | — | — | — | — | — | X | 97 | 61 |
| MP3A7SRA | — | — | — | — | — | — | — | X | 61 |
| MP4C10SR | — | — | — | — | — | — | — | — | X |
| MP4D5BR | — | — | — | — | — | — | — | — | — |
| MP3F1SRA | — | — | — | — | — | — | — | — | — |
| MP6D6BR | — | — | — | — | — | — | — | — | — |
| MP6B1BR | — | — | — | — | — | — | — | — | — |
| MP6A8BR | — | — | — | — | — | — | — | — | — |
| MP6B12BR | — | — | — | — | — | — | — | — | — |
| MP6C11BR | | | | | | | | | |
| MP6B10BR | | | | | | | | | |

| | % Homology | | | | | | | |
|---|---|---|---|---|---|---|---|---|
| | MP4D5BR | MP3F1SRA | MP6D6BR | MP6B1BR | MP6A8BR | MP6B12BR | MP6C11BR | MP6B10BR |
| MP3D2SRA | 72 | 65 | 68 | 67 | 66 | 67 | 76 | 70 |
| MP3A3SR | 73 | 65 | 67 | 67 | 65 | 66 | 77 | 71 |
| MP3C5SR | 67 | 60 | 74 | 63 | 60 | 63 | 70 | 64 |
| MP3C1SR | 67 | 59 | 73 | 63 | 60 | 62 | 70 | 65 |
| MP3G8SR | 66 | 60 | 73 | 63 | 61 | 63 | 71 | 64 |
| MP3D2BR | 65 | 58 | 73 | 64 | 60 | 63 | 68 | 67 |
| MP3H6SRA | 71 | 69 | 71 | 71 | 68 | 70 | 82 | 70 |
| MP3B4SRA | 71 | 69 | 71 | 71 | 68 | 70 | 82 | 70 |
| MP4E4BR | 72 | 70 | 71 | 71 | 68 | 70 | 80 | 71 |
| MP4H8SR | 70 | 67 | 69 | 70 | 67 | 70 | 79 | 71 |
| MP2F6SR | 69 | 66 | 67 | 69 | 68 | 67 | 78 | 69 |
| MP3D1BR | 68 | 66 | 67 | 71 | 69 | 69 | 79 | 70 |
| MP2B5BR | 63 | 84 | 65 | 63 | 63 | 62 | 70 | 65 |
| MP2C1BR | 61 | 85 | 65 | 64 | 63 | 62 | 70 | 65 |
| MP4A12SR | 61 | 84 | 64 | 63 | 63 | 62 | 70 | 65 |
| MP3F4SRA | 72 | 63 | 67 | 68 | 65 | 65 | 76 | 71 |
| MP3D3BR | 70 | 64 | 66 | 66 | 64 | 64 | 75 | 69 |
| MP3E5BR | 72 | 64 | 67 | 68 | 65 | 66 | 77 | 71 |
| MP3C7SRA | 72 | 64 | 68 | 68 | 66 | 66 | 78 | 71 |
| MP2F1BR | 70 | 64 | 68 | 65 | 64 | 64 | 74 | 67 |
| MP2C5BR | 69 | 63 | 67 | 64 | 62 | 63 | 73 | 67 |
| MP2C10BR | 67 | 66 | 69 | 68 | 64 | 68 | 74 | 73 |
| MP2G5SR | 67 | 65 | 67 | 66 | 64 | 66 | 73 | 73 |
| MP3B1SRA | 67 | 60 | 67 | 69 | 68 | 69 | 69 | 65 |
| MP2F10SR | 67 | 65 | 71 | 66 | 65 | 67 | 77 | 68 |
| MP3A7SRA | 68 | 63 | 71 | 65 | 65 | 67 | 77 | 69 |
| MP4C10SR | 64 | 58 | 65 | 64 | 63 | 66 | 66 | 63 |
| MP4D5BR | X | 64 | 69 | 68 | 65 | 67 | 76 | 73 |
| MP3F1SRA | — | X | 65 | 64 | 64 | 63 | 71 | 68 |
| MP6D6BR | — | — | X | 70 | 65 | 70 | 77 | 73 |
| MP6B1BR | — | — | — | X | 78 | 81 | 76 | 71 |
| MP6A8BR | — | — | — | — | X | 75 | 74 | 66 |
| MP6B12BR | — | — | — | — | — | X | 73 | 68 |
| MP6C11BR | | | | | | | X | 77 |
| MP6B10BR | | | | | | | | X |

1. An anti-IFN-gamma polypeptide comprising at least one anti-IFN-gamma single domain antibody.

2. An anti-IFN-gamma polypeptide according to claim 1, wherein at least one anti-IFN-gamma single domain antibody, is a Camelidae VHH antibody.

3. An anti-IFN-gamma polypeptide according to claim 1 wherein at least one single domain antibody corresponds to a sequence represented by any of SEQ ID NOs: 1 to 35.

4. An anti-IFN-gamma polypeptide according to claim 1 further comprising at least one single domain antibody directed against a serum protein.

5. An anti-IFN-gamma polypeptide according to claim 4 wherein the serum protein is any of serum albumin, serum immunoglobulins, thyroxine-binding protein, transferrin, or fibrinogen.

6. An anti-IFN-gamma polypeptide according to claim 4 wherein the anti-serum protein single domain antibody correspond to a sequence represented by any of SEQ ID NOs: 36 to 39 and 62 to 74.

7. An anti-IFN-gamma polypeptide according to claim 4 corresponding to a sequence represented by any of SEQ ID NOs: 40 to 42.

8. An anti-IFN-gamma polypeptide according to claim 1 further comprising at least one single domain antibody selected from the group consisting of anti-TNF-alpha single domain antibody, anti-TNF-alpha receptor single domain antibody and anti-IFN-gamma receptor single domain antibody.

9. An anti-IFN-gamma polypeptide according to claim 1, wherein the number of single domain antibodies directed against IFN-gamma is at least two.

10. An anti-IFN-gamma polypeptide according to claim 9 corresponding to a sequence represented by any of SEQ ID NOs: 59 to 61.

11. An anti-IFN-gamma polypeptide according to claim 1, wherein at least one single domain antibody is a humanized Camelidae VHHs.

12. A composition comprising an anti-IFN-gamma polypeptide according to claim 1 together with at least one single domain antibody from the group consisting of anti-TNF-alpha single domain antibody, anti-TNF-alpha receptor single domain antibody and anti-IFN-gamma receptor single domain antibody, for simultaneous, separate or sequential administration to a subject.

13. An anti-IFN-gamma polypeptide according to claim 8 wherein at least one anti-TNF-alpha single domain antibody correspond to a sequence represented by any of SEQ ID NOs: 43 to 58.

14.-15. (canceled)

16. An anti-IFN-gamma polypeptide according to claim 4 wherein said single domain antibodies are Camelidae VHHs.

17. A nucleic acid encoding an anti-IFN-gamma polypeptide according to claim 1.

18. A method of identifying an agent that modulates the binding of an anti-IFN-gamma polypeptide to IFN-gamma comprising the steps of:
(a) contacting an anti-IFN-gamma polypeptide of claim 1 with a target that is IFN-gamma, in the presence and absence of a candidate modulator under conditions permitting binding between said polypeptide and target, and
(b) measuring the binding between the polypeptide and target of step (a), wherein a decrease in binding in the presence of said candidate modulator, relative to the binding in the absence of said candidate modulator, identifies said candidate modulator as an agent that modulates the binding of an anti-IFN-gamma polypeptide and IFN-gamma.

19. A method of identifying an agent that modulates IFN-gamma-mediated disorders through the binding of an anti-IFN-gamma polypeptide to IFN-gamma comprising:
(a) contacting an anti-IFN-gamma polypeptide of claim 1 with a target that is IFN-gamma, in the presence and absence of a candidate modulator under conditions permitting binding between said polypeptide and target, and
(b) measuring the binding between the polypeptide and target of step (a), wherein a decrease in binding in the presence of said candidate modulator, relative to the binding in the absence of said candidate modulator, identifies said candidate modulator as an agent that modulates IFN-gamma-mediated disorders.

20. A method of identifying an agent that modulates the binding of IFN-gamma to its receptor through the binding of an anti-IFN-gamma polypeptide to IFN-gamma comprising:
(a) contacting an anti-IFN-gamma polypeptide of claim 1 with a target that is IFN-gamma, in the presence and absence of a candidate modulator under conditions permitting binding between said polypeptide and target, and
(b) measuring the binding between the polypeptide and target of step (a), wherein a decrease in binding in the presence of said candidate modulator, relative to the binding in the absence of said candidate modulator, identifies said candidate modulator as an agent that modulates the binding of IFN-gamma to its receptor.

21. A kit for screening for agents that modulate IFN-gamma-mediated disorders comprising an anti-IFN- gamma polypeptide of claim 1 and IFN-gamma.

22.-25. (canceled)

26. A method for treating and/or preventing and/or alleviating disorders relating to inflammatory reactions comprising administering to a subject in need of such treatment an effective amount of the anti-IFN-gamma polypeptide of claim 1.

27. (canceled)

28. A method for treating and/or preventing and/or alleviating the symptoms of disorders requiring the delivery of an IFN-gamma modulating polypeptide comprising administering to a subject in need of such treatment an effective amount of the anti-IFN-gamma polypeptide of claim 1 that passes through the gastric environment without being inactivated.

29. (canceled)

30. A method for treating and/or preventing and/or alleviating the symptoms of disorders requiring the delivery of an IFN-gamma modulating polypeptide comprising administering to a subject in need of such treatment an effective amount of the anti-IFN-gamma polypeptide of claim 1.

31. (canceled)

32. A method for treating and/or preventing and/or alleviating the symptoms of disorders requiring the delivery of a therapeutic compound to the upper respiratory tract and lung comprising administering to a subject in need of such treatment an effective amount of the anti-IFN-gamma polypeptide of claim 1.

33. (canceled)

34. A method for treating and/or preventing and/or alleviating the symptoms of disorders requiring the delivery of a IFN-gamma modulator comprising administering to a subject in need of such treatment an effective amount of the anti-IFN-gamma polypeptide of claim 1 wherein said disorder increases the permeability of the intestinal mucosa.

35. (canceled)

36. A method for treating and/or preventing and/or alleviating the symptoms of disorders requiring the delivery of a IFN-gamma modulator comprising administering to a subject in need of such treatment an effective amount of the anti-IFN-gamma polypeptide of claim 1 that passes through the tissues beneath the tongue.

37. (canceled)

38. A method for treating and/or preventing and/or alleviating the symptoms of disorders requiring the delivery of a IFN-gamma modulator comprising administering to a subject in need of such treatment an effective amount of the anti-IFN-gamma polypeptide of claim 1 that passes through the skin.

39. A method according to any of claims 26, 28, 30, 32, 34, 36, and 38, wherein said disorders are any of inflammation, rheumatoid arthritis, Crohn's disease, ulcerative colitis, inflammatory bowel syndrome, multiple sclerosis, Addison's disease, Autoimmune hepatitis, Autoimmune parotitis, Diabetes Type I, Epididymitis, Glomerulonephritis, Graves' disease, Guillain-Barre syndrome, Hashimoto's disease, Hemolytic anemia, Systemic lupus erythematosus, Male infertility, Multiple sclerosis, Myasthenia Gravis, Pemphigus, Psoriasis, Rheumatic fever, Rheumatoid arthritis, Sarcoidosis, Scleroderma, Sjogren's syndrome, Spondyloarthropathies, Thyroiditis, and Vasculitis.

40. A composition comprising the anti-IFN-gamma polypeptide of claim 1, and a suitable pharmaceutical vehicle.

41. A method of diagnosing a disorder characterised by the dysfunction of IFN-gamma comprising:
(a) contacting a sample with an anti-IFN-gamma polypeptide of claim 1,
(b) detecting binding of said polypeptide to said sample, and
(c) comparing the binding detected in step (b) with a standard, wherein a difference in binding relative to said sample is diagnostic of a disorder characterised by dysfunction of IFN-gamma.

42.-43. (canceled)

44. A method for purification of IFN-gamma, comprising contacting a sample with the anti-IFN-gamma polypeptide of claim 1.

45. A method for inhibiting the interaction between IFN-gamma and one or more IFN-gamma receptors, comprising contacting IFN-gamma with the anti-TNF-alpha polypeptide of claim 1.

46. A method for producing an anti-IFN-gamma polypeptide of claim 1 comprising the steps of:
(a) obtaining double stranded DNA encoding a Camelidae VHH directed to IFN-gamma,
(b) cloning and expressing the DNA selected in step (b).

47. A method of producing an anti-IFN-gamma polypeptide of claim 1 comprising:
(a) culturing host cells comprising nucleic acid that encode an anti-IFN-gamma polypeptide of claim 1, under conditions allowing the expression of the polypeptide, and,
(b) recovering the produced polypeptide from the culture.

48. A method according to claim 47, wherein said host cells are bacterial or yeast.

49. (canceled)
